(19) United States
(12) Patent Application Publication
(10) Pub. No.: US 2008/0050419 A1
(43) Pub. Date: Feb. 28, 2008

(54) Epoxy-containing poly(ester amides) and method of use

(75) Inventors: Ramaz Katsarava, Tbilisi (GE); David Tugushi, Tbilisi (GE); Nino Zavradashvili, Anaga village (GE); Zaza D. Gomurashvili, La Jolla, CA (US)

Correspondence Address: DLA PIPER US LLP, 4365 EXECUTIVE DRIVE, SUITE 1100, SAN DIEGO, CA, 92121-2133, US

(73) Assignee: MediVas, LLC, San Diego, CA (US)

(21) Appl. No.: 11/893,719

(22) Filed: Aug. 16, 2007

Related U.S. Application Data
(60) Provisional application No. 60/838,699, filed on Aug. 18, 2006.

(51) Int. Cl. C08J 3/28; A61K 9/00

(52) U.S. Cl. 424/426; 424/484; 523/113; 528/363

(57) ABSTRACT

The invention provides aliphatic epoxy-containing PEA polymer compositions with film-forming properties. The aliphatic epoxy di-acids used in the invention PEA compositions include non-toxic fatty aliphatic epoxy homologs. A second, C-protected L-lysine-based monomer can be introduced into the polymer to provide additional chain flexibility. The invention PEA polymer compositions are useful for delivery of bioactive agents when administered internally or used in the manufacture of implantable medical devices. Biodegradable hydrogels can be made using the invention epoxy-containing PEAs.

### RELATED APPLICATIONS

This application relies for priority under 35 U.S.C. §119(e) on U.S. Ser. No. 60/838,699, filed Aug. 18, 2006, which is incorporated herein by reference.

### FIELD OF THE INVENTION

The invention relates, in general, to drug delivery systems and, in particular, to polymer delivery compositions that incorporate alpha-amino acids and epoxy-functionalities into a biodegradable polymer backbone.

### BACKGROUND OF THE INVENTION

Regular AA-BB-type bio-analogous poly(ester amides) (PEAs), consisting of nontoxic building blocks, such as hydrophobic α-amino acids, aliphatic diols and di-carboxylic acids have been proven to be important materials for biomedical applications because of their excellent blood and tissue compatibility (K. DeFife et al. Transcatheter Cardiovascular Therapeutics—TCT 2004 Conference. Poster presentation. Washington D.C. 2004) and biologic degradation profiles (G. Tsitlanadze, et al. J. Biomater. Sci. Polymer Edn. (2004). 15:1-24). Controlled enzymatic degradation and low nonspecific degradation rates of PEAs make them attractive for drug delivery applications.

These properties of PEAs provide advantages over widely used aliphatic polyesters, such as polylactic acid (PLA) and polyglycolic acid (PGA). Aliphatic ester-groups in macromolecules of PLA and PGA contribute to rapid hydrolytic degradation rates, but polymer surfaces are known to display poor adhesion and cell growth, which properties are important indicators of cell-biomaterial interactions (Cook, A D, et al. J. Biomed. Mater. Res., (1997). 35: 513-523).

PEAs have high potential for various biomedical applications due to lateral—COOH groups, introduced from L-lysine moieties. Free carboxylates also represent chemical attachment sites for drugs and bio-active substances and have been successfully used for the covalent attachment of physiologically active nitric oxide derivatives like 4-amino TEMPO. (U.S. Pat. No. 6,503,538). These functional PEAs, however, are not suitable for preparing mixed anhydrides by interaction, for example, with methacrylic anhydride, due to the well known tendency of mixed anhydrides of N^{α}-acyl amino acids to form azlactons and racemic mixtures (Greenstein J. P. and Vinitz M., Chemistry of the amino acids. John Willey & Sons, Inc., New York-London, 1961) as well as to undergo the Dakin-West reaction (Iwakura Y. et al. J. Org. Chem., (1967)] 32, 440).

Therefore, an alternative innovative approach is necessary for introducing functional groups into the biodegradable PEA backbone to provide active polymers useful in designing three-dimensional biodegradable hybrid networks for tissue engineering and other applications.

Biodegradable PEAs with unsaturated moieties in main chain were recently reported by Guo et al. (J. Polym. Sci. Part A. Polym. Chem. (2005) 43:1463-1477), where unsaturated fumaric acid and 2-butene-1,4-diol were implemented as building blocks.

The conventional melt polycondensation of monomers having reactive functional groups causes cross-linking or thermal degradation of functional groups at elevated temperature. Therefore the need exists for new functionalized PEAs and better methods for synthesis of such functionalized PEAs, such as high molecular weight, linear PEAs containing epoxy groups in the polymer backbone. The need also exists in the art for new chemical transformations of such epoxy-containing PEAs to produce polymers useful for various medical applications.

### SUMMARY OF THE INVENTION

The present invention is based on the discovery of a new class of linear functional condensation poly(ester-amides)s with epoxy moieties in the macromolecular backbone.

The invention provides high molecular weight, linear PEAs containing epoxy moieties in the polymer backbone formed by low temperature active polycondensation of epoxy-containing bis-electrophiles—active diesters with di-p-toluenesulfonic acid salts of bis-(α-amino acid)-α,ω-alkylene diesters. Chemical transformations of such epoxy-containing PEAs are also provided.

Accordingly in one embodiment, the invention provides biodegradable polymer compositions containing at least one or a blend of a poly(ester amide) (PEA) polymers having a chemical formula described by general structural formula (I), 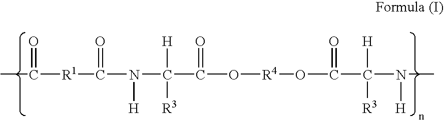
wherein n ranges from about 15 to about 150; R¹ in at lest one individual n unit is epoxy-(C₂-C₁₂)alkylene, while additional R¹s are independently selected from (C₂-C₂₀)alkylene, (C₂-C₂₀)alkenylene, α,ω-bis(4-carboxyphenoxy)-(C₁-C₈) alkane, 3,3′-(alkanedioyldioxy)dicinnamic acid or 4,4′-(alkanedioyldioxy)dicinnamic acid, α,ω-alkylene dicarboxylates of formula (III) below or saturated or unsaturated residues of therapeutic di-acids; whereas R⁵ and R⁶ in formula (III) are independently selected from (C₂-C₁₂)alkylene or (C₂-C₁₂)alkenylene; the R³s in individual n units are independently selected from the group consisting of hydrogen, (C₁-C₆)alkyl, (C₂-C₆)alkenyl, (C₂-C₆)alkynyl, (C₆-C₁₀)aryl (C₁-C₆)alkyl, and —(CH₂)₂SCH₃; and R⁴ is independently selected from the group consisting of (C₂-C₂₀)alkylene, (C₂-C₂₀)alkenylene, (C₂-C₈)alkyloxy, (C₂-C₂₀)alkylene, bicyclic-fragments of 1,4:3,6-dianhydrohexitols of structural formula (II), saturated or unsaturated therapeutic diol residues, and combinations thereof;

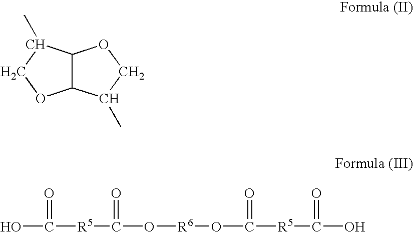

or PEA polymers having a chemical formula described by structural formula (IV): 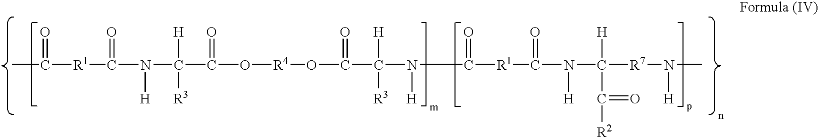
wherein n ranges from about 15 to about 150, m ranges about 0.1 to 0.9; p ranges from about 0.9 to 0.1; R¹ in at least one individual n or m unit is epoxy-(C₂-C₁₂)alkylene, while additional R¹s are independently selected from (C₂-C₂₀)alkylene and (C₂-C₂₀)alkenylene, α,ω-bis(4-carboxyphenoxy)-(C₁-C₈)alkane, 3,3′-(alkanedioyldioxy)dicinnamic acid, 4′-(alkanedioyldioxy)dicinnamic acid, or α,ω-alkylene dicarboxylates of structural formula (III) or saturated or unsaturated residues of therapeutic di-acids;

wherein R⁵ and R⁶ in formula (III) are independently selected from (C₂-C₁₂)alkylene or (C₂-C₁₂)alkenylene; each R² is independently hydrogen, (C₁-C₁₂)alkyl, (C₆-C₁₀)aryl or a protecting group; the R³s in individual m monomers are independently selected from the group consisting of hydrogen, (C₁-C₆)alkyl, (C₂-C₆)alkenyl, (C₂-C₆)alkynyl, (C₆-C₁₀)aryl (C₁-C₆)alkyl, and —(CH₂)₂SCH₃; R⁴ is independently selected from the group consisting of (C₂-C₂₀)alkylene, (C₂-C₂₀)alkenylene, (C₂-C₈)alkyloxy (C₂-C₂₀)alkylene, bicyclic-fragments of 1,4:3,6-dianhydrohexitols of structural formula (II), and residues of saturated or unsaturated therapeutic diols and combinations thereof; and R⁷ is independently (C₂-C₂₀)alkyl or (C₂-C₂₀)alkenyl.

In another embodiment, the invention presents methods for delivering a bioactive agent to a subject by implanting at an interior body site an invention epoxy-containing PEA composition with at least one bioactive agent dispersed within the polymer. The composition will slowly biodegrade, for example completely. During biodegradation of the composition, e.g. in particles made thereof, or a medical device containing a coating of the composition, the bioactive agent(s) dispersed in the polymer will be slowly released to tissue surrounding a site of implantation, for example to promote healing and alleviate pain therein.

### BRIEF DESCRIPTION OF THE FIGURES

FIG. 1 is a graph showing swelling of hybrid hydrogels obtained on the basis of content of hybrid methacryloyl dextran (MaDX) and unsaturated epoxy-containing PEA (PEA I.6).

FIG. 2 is a graph showing the time for in vitro biodegradation (weigh loss in mg/cm²) of epoxy-containing PEA t-ES-L-Phe-6, (I.1) in enzyme solution (0.2 M phosphate buffer, pH=7.4, t=25° C.) under the action of a) α-chymotrypsin (4 mg/10 mL) and b) lipase (4 mg/10 mL).

FIG. 3 is a graph showing time for in vitro biodegradation (weigh loss in mg/cm²) of a PEA that does not contain an epoxy functionality, 8-L-Phe-6 in enzyme solution (0.2 M phosphate buffer, pH=7.4, t=25° C.) under the action of a) α-chymotrypsin (4 mg/10 mL) or b) lipase (4 mg/10 mL).

FIG. 4 is a graph showing the time for α-Chymotrypsin catalyzed in vitro biodegradation of epoxy-containing PEA t-ES-L-Phe-6 (I.1): curve 0=the starting sample of PEA, curves 1-4=the samples of PEA, heated at 120° C. for 1 h, 6 h, 12 h and 24 h, respectively.

FIG. 5 is a graph showing the time for lipase catalyzed in vitro biodegradation of epoxy-containing PEA t-ES-L-Phe-6 (I.2): curve 0=the starting sample of PEA, curves 1-4=the samples of PEA, heated at 120° C. for 1 h, 6 h, 12 h and 24 h, respectively.

### DETAILED DESCRIPTION OF THE INVENTION

The invention is based on the discovery of a new class of functional poly(ester amides) (PEAs) which feature epoxy groups in the polymer backbone. Epoxy functionalities in the invention epoxy-containing PEAs are introduced in the form of aliphatic epoxy-di-acids (as bis-electrophilic monomers). Synthesis of the invention expoxy-containing PEAs is carried out by active polycondensation methods, wherein active esters of epoxy-di-acids are reacted with bis(α-aminoacyl)-α,ω-alkylene-diesters in solution in the presence of tertiary amine.

To illustrate the invention, active polycondensation reactions were carried out at mild temperatures (≦60° C.) in polar aprotic solvents (DMF, DMAc). The resulting reaction solutions are very viscous; however, formation of gel during the reaction by undesired intermolecular reactions was not observed in any of the cases studied. After precipitation from the reaction mixture in water, new polymers—epoxy-containing PEAs with rather high viscosities (Table. 2.1) were obtained and good film-forming properties of the polymers were observed. The remarkable feature of this process consists in the absence of gel formation during polycondensation, which demonstrates a virtual lack of intermolecular reaction between epoxy groups in the polymeric backbones and terminal amino groups of the growing chains under the reaction conditions.

Accordingly in one embodiment, the invention provides biodegradable polymer compositions comprising at least one or a blend of PEA polymers having a chemical formula described by general structural formula (I), 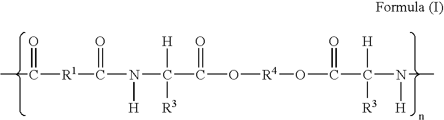
wherein n ranges from about 15 to about 150; R¹ in at lest one individual n unit is epoxy-(C₂-C₁₂)alkylene, while additional R¹s are independently selected from (C₂-C₂₀)alkylene, (C₂-C₂₀)alkenylene, α,ω-bis(4-carboxyphenoxy)-(C₁-C₈)alkane, 3,3′-(alkanedioyldioxy)dicinnamic acid or 4,4′-(alkanedioyldioxy)dicinnamic acid, α,ω-alkylene dicarboxylates of formula (III) below or saturated or unsaturated residues of therapeutic di-acids; whereas R⁵ and R⁶ in Formula (III) are independently selected from (C₂-C₁₂)alkylene or (C₂-C₁₂)alkenylene; the R³s in individual n units are independently selected from the group consisting of hydrogen, (C₁-C₆)alkyl, (C₂-C₆)alkenyl, (C₂-C₆)alkynyl, (C₆-C₁₀)aryl (C₁-C₆)alkyl, and —(CH₂)₂SCH₃; and R⁴ is independently selected from the group consisting of (C₂-C₂₀)alkylene, (C₂-C₂₀)alkenylene, (C₂-C₈)alkyloxy, (C₂-C₂₀)alkylene, bicyclic-fragments of 1,4:3,6-dianhydrohexitols of structural formula (II), saturated or unsaturated therapeutic diol residues, and combinations thereof;

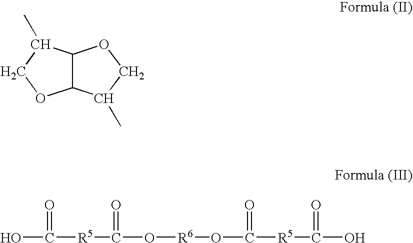

or a PEA polymer having a chemical formula described by structural formula (IV): 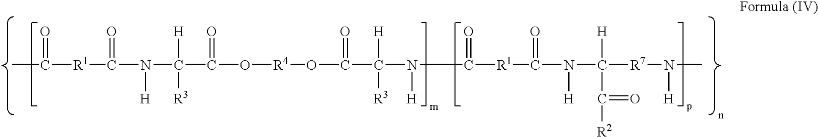
wherein n ranges from about 15 to about 150, m ranges about 0.1 to 0.9; p ranges from about 0.9 to 0.1; R¹ in at lest one individual n or m unit is epoxy-(C₂-C₁₂)alkylene, while additional R¹s are independently selected from (C₂-C₂₀)alkylene and (C₂-C₂₀)alkenylene, α,ω-bis(4-carboxyphenoxy)-(C₁-C₈)alkane, 3,3′-(alkanedioyldioxy)dicinnamic acid, 4,4′-(alkanedioyldioxy)dicinnamic acid, or α,ω-alkylene dicarboxylates of structural formula (III) or saturated or unsaturated residues of therapeutic di-acids;

wherein R⁵ and R⁶ in Formula (III) are independently selected from (C₂-C₁₂)alkylene or (C₂-C₁₂)alkenylene; each R² is independently hydrogen, (C₁-C₁₂)alkyl, (C₆-C₁₀)aryl or a protecting group; the R³s in individual m monomers are independently selected from the group consisting of hydrogen, (C₁-C₆)alkyl, (C₂-C₆)alkenyl, (C₂-C₆)alkynyl, (C₆-C₁₀)aryl (C₁-C₆)alkyl, and —(CH₂)₂SCH₃; and R⁴ is independently selected from the group consisting of (C₂-C₂₀)alkylene, (C₂-C₂₀)alkenylene, (C₂-C₈)alkyloxy, (C₂-C₂₀)alkylene, bicyclic-fragments of 1,4:3,6-dianhydrohexitols of structural formula (II), and residues of saturated or unsaturated therapeutic diols and combinations thereof; and R⁷ is independently (C₂-C₂₀)alkyl or (C₂-C₂₀)alkenyl. For example, R⁷ can be (C₃-C₆)alkyl or (C₃-C₆)alkenyl, but is preferably —(CH₂)₄—.

A typical protecting group for use in the invention polymers is t-butyl, or others as are known in the art. The bicyclic-fragments of 1,4:3,6-dianhydrohexitols, also called “sugar-diols,” are derived from starch, such as D-glucitol (isosorbide (1,4:3,6-dianhydrosorbitol)), D-mannitol, or L-iditol.

The “n” monomers in the invention epoxy-containing PEA polymers of structural formula (I) can be identical, in which case the polymer is referred to herein as a “homo-polymer.” Alternatively, the “n” monomers in the invention epoxy-containing PEA polymers of structure (I) can be different, being fabricated using different combinations of building blocks (i.e., diols, di-acids and cc-amino acids), in which case the polymer is referred to herein as a “co-polymer”. In the invention epoxy-containing PEA polymers of formula (IV), which include a second monomer “p,” the “m” monomers can also be either identical or different.

As used herein, the term “residue of a therapeutic di-acid” means a portion of a dicarboxylic-acid with therapeutic properties, as described herein, that excludes the two carboxyl groups of the di-acid. As used herein, the term “residue of a therapeutic diol” means a portion of a diol with therapeutic properties, as described herein, which excludes the two hydroxyl groups of the diol. The corresponding di-acid or diol containing the “residue” thereof is used in synthesis of the co-polymer compositions. The residue of the therapeutic di-acid or diol is reconstituted in vivo (or under similar conditions of pH, aqueous media, and the like) to the corresponding therapeutic diol or di-acid upon release from the polymer composition by biodegradation in a controlled manner that depends upon the properties of the α,ω-bis(4-carboxyphenoxy)alkane-containing polymer used in the composition, which properties are as described herein, for example in the Examples.

As used herein, the terms “α-amino acid-containing”, and “α-amino acid” mean a chemical compound containing an amino group, a carboxyl group and an R³ group as defined herein. As used herein, the terms “biological α-amino acid-containing” and “biological α-amino acid” mean the α-amino acid(s) used in synthesis is phenylalanine, leucine, glycine, alanine, valine, isoleucine, methionine, proline, or a mixture thereof. When the R³s are —(CH₂)₃—, the α-amino acid is analogous to pyrrolidine-2-carboxylic acid.

As used herein the term “bioactive agent” means a bioactive agent as disclosed herein that is not incorporated into the polymer backbone, but is dispersed within the alkylene di-acid containing PEA polymer. One or more such bioactive agents may optionally be included in the invention epoxy-containing PEA polymer compositions. As used herein to refer to bioactive agents, the term “dispersed” means the bioactive agents are dispersed into mixed with, dissolved in, homogenized with, and/or covalently bound to an invention polymer composition, for example attached to a functional group in the PEA polymer of the composition or to the surface of a polymer particle or medical device made using the invention epoxy-containing PEA composition.

Use of a residue of a saturated or unsaturated alkyl diol in the monomers provides elongation properties of the resulting polymer. A second “p” monomer, optionally, L-lysine-based, can be included in an invention epoxy-containing PEA polymer to introduce an additional reactive group (such as a pending C-terminus), which can be modified to further control the thermo-mechanical properties of the polymer.

The invention biodegradable polymers containing unsaturated groups have additional potential for various applications. For example, unsaturated groups can be converted into another functional group, such as an alcohol, that is useful for further modification, such as attachment of a bioactive agent. The crosslinking of polymers containing unsaturated groups can enhance thermal and mechanical properties of the polymer, for example as is illustrated herein.

Like other PEA polymers, the invention epoxy-containing PEA polymer compositions can be used to deliver in vivo at least one bioactive agent that is dispersed in the polymer of the composition. The invention epoxy-containing PEA polymer compositions biodegrade in vivo by enzymatic action so as to release the at least one bioactive agent(s) from the polymer in a controlled manner over time. Moreover, since theoretically the bis(α-amino acid)-diol-diester co-monomers in the invention epoxy-containing PEA polymers may each contain a different one of the multiple amino acids disclosed herein in each bis(α-amino acid) building block, the invention PEA polymer compositions may break down to produce from one to multiple different of such α-amino acids.

The terms, “biodegradable” and “biocompatible” as used herein to describe the invention epoxy-containing PEA polymer compositions means the polymer is capable of being broken down into innocuous products in the normal functioning of the body. This is particularly true when the amino acids used in fabrication of the PEA polymers are biological L-α-amino acids. These PEA polymer compositions include ester groups hydrolyzable by esterases and enzymatically cleavable amide linkages that provide biodegradability, and are typically chain terminated, predominantly with amino groups. Alternatively, the amino termini of the polymers can be acetylated or otherwise capped by conjugation to any other acid-containing, biocompatible molecule, to include without restriction organic acids, bioinactive biologics, and bioactive agents as described herein. In one embodiment, the entire polymer composition, and any particles, coating or medical device made thereof, is substantially biodegradable and biocompatible.

In one alternative, at least one of the α-amino acids used in fabrication of the invention epoxy-containing PEA polymers is a biological α-amino acid. For example, when the R³s are CH₂Ph, the biological α-amino acid used in synthesis is L-phenylalanine. In alternatives wherein the R³s are CH₂—CH(CH₃)₂, the polymer contains the biological α-amino acid, L-leucine. By varying the R³s within co-monomers as described herein, other biological α-amino acids can also be used, e.g., glycine (when the R³s are H), alanine (when the R³s are CH₃), valine (when the R³s are CH(CH₃)₂), isoleucine (when the R³s are CH(CH₃)—CH₂—CH₃) or methionine (when the R³s are —(CH₂)₂SCH₃), and mixtures thereof. When the R³s are —(CH₂)₃— (as in 2-pyrrolidinecarboxylic acid), a biological α-imino acid proline can be used. In yet another alternative embodiment, all of the various α-amino acids contained in the invention epoxy-containing PEA polymers are biological α-amino acids, as described herein.

The term “aryl” is used with reference to structural formulas herein to denote a phenyl radical or an ortho-fused bicyclic carbocyclic radical having about nine to ten ring atoms in which at least one ring is aromatic. In certain embodiments, one or more of the ring atoms can be substituted with one or more of nitro, cyano, halo, trifluoromethyl, or trifluoromethoxy. Examples of aryl include, but are not limited to, phenyl, naphthyl, and nitrophenyl.

The term “alkenylene” is used with reference to structural formulas herein to mean a divalent branched or unbranched hydrocarbon chain containing at least one unsaturated bond in the main chain or in a side chain.

Further, the epoxy-containing PEA polymer compositions suitable for use in the practice of the invention bear functionalities that allow the option of covalent attachment of bioactive agent(s) to the polymer. For example, a polymer bearing free carboxyl groups can readily react with an amino moiety, thereby covalently bonding a peptide to the polymer via the resulting amide group. As will be described herein, the biodegradable polymer and a bioactive agent may contain numerous complementary functional groups that can be used to covalently attach the bioactive agent to the biodegradable polymer.

Further examples of PEA polymers related to those contemplated for use in the practice of the invention and methods of synthesis include those set forth in U.S. Pat. Nos. 5,516,881; 5,610,241; 6,476,204; and 6,503,538; and in U.S. application Ser. Nos. 10/096,435; 10/101,408; 10/143,572; 10/194,965 and 10/362,848.

In certain embodiments, particles, a coating, or a medical device made from or containing the invention epoxy-containing PEA polymer composition, as described herein, plays an active role in the treatment processes at the site of implant or use by holding the polymer and any bioactive agents dispersed therein at the site for a period of time sufficient to allow the subject's endogenous processes to slowly release particles or polymer molecules from the composition. Meanwhile, the subject's endogenous processes biodegrade the polymer so as to release bioactive agents dispersed in the polymer. The fragile optional bioactive agents are protected by the more slowly biodegrading polymer to increase half-life and persistence of the bioactive agent(s) locally at the site of use, e.g., implant.

Uptake of the polymer with bioactive agent is safe: studies have shown that the subject can metabolize/clear the polymer degradation products. The invention epoxy-containing PEA polymer compositions are, therefore, substantially non-inflammatory to the subject both at the site of implant and systemically, apart from any trauma caused by implantation itself.

The invention biodegradable homo-polymer and co-polymer compositions preferably have weight average molecular weights ranging from 15,000 to 600,000 Daltons; these polymers and copolymers typically have inherent viscosities at 25° C., determined by standard viscosimetric methods, ranging from 0.15 to 3.5, preferably ranging from 0.4 to 2.0.

The molecular weights and polydispersities herein are determined by gel permeation chromatography (GPC) using polystyrene standards. More particularly, number and weight average molecular weights (Mₙ and M_{w}) are determined, for example, using a Model 510 gel permeation chromatographer (Water Associates, Inc., Milford, Mass.) equipped with a high-pressure liquid chromatographic pump, a Waters 486 UV detector and a Waters 2410 differential refractive index detector. Solution of 0.1% LiBr in N,N-dimethylacetamide (DMAc), or 0.1% LiCl in N,N-dimethylformamide (DMF) is used as the eluent (1.0 mL/min). The polystyrene (PS) or Polyethyleneglycol (PEG) standards, with narrow molecular weight distribution were used for calibration of GPC curves.

Methods for making PEA polymers containing α-amino acids in the general formula are well known in the art. For example, for the embodiment of the polymer of formula (I), a α-amino acid can be converted into a bis(α-amino acid)-diol-diester monomer, for example, by condensing the α-amino acid with a diol as described herein. As a result, ester bonds are formed. Then, the bis(α-amino acid)-diol-diester is entered into a polycondensation reaction with a di-acid, such as sebacic acid, or α,ω-bis(4-carboxyphenoxy)alkanoic di-acid, to obtain the final polymer having both ester and amide bonds. Alternatively, instead of the di-acid, an activated di-acid derivative, e.g., di-(p-nitrophenyl)ester, can be used for polymers of chemical structure (I).

More particularly, synthesis of the unsaturated poly(ester-amide)s (UPEAs) useful as biodegradable polymers of the structure (I) or (IV) as described above will be described wherein: 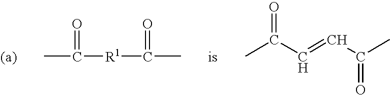
for example, and/or (b) R³ is —CH₂—CH═CH—CH₂—. In cases where (a) is present and (b) is not present, R³ can be —C₄H₈— or —C₆H₁₂—. In cases where (a) is not present and (b) is present, R¹ can be —C₄H₈— or —C₈H₁₆—.

The UPEAs can be prepared by solution polycondensation of either (1) di-p-toluene sulfonic acid salt of bis(α-amino acid)diesters, comprising at least 1 double bond in the diol residue, a di-p-toluene sulfonic acid salt of a bis(α-amino acid)-alkylene-diesters, comprising a diol of structural formula (III), and di-(p-nitrophenyl)esters of saturated dicarboxylic acid or (2) two di-p-toluene sulfonic acid salt of bis(α-amino acid)alkylene-diesters, comprising no double bonds in the diol residues, and di-(p-nitrophenyl)ester of unsaturated dicarboxylic acid or (3) two di-p-toluene sulfonic acid salts of bis(α-amino acid)-diol-diesters, comprising at least one double bond in one of the diol residues in the polymer general structural formula, the other diol residue having structural formula (III), and di-nitrophenyl esters of unsaturated dicarboxylic acids.

Salts of p-toluene sulfonic acid are known for use in synthesizing polymers containing amino acid residues. The aryl sulfonic acid salts are used instead of the free base because the aryl sulfonic acid salts of bis(α-amino acid)-alkylene-diesters are easily purified through recrystallization and render the amino groups as stable ammonium tosylates throughout workup. In the polycondensation reaction, the nucleophilic amino group is readily revealed through the addition of an organic base, such as triethylamine, so the polymer product is obtained in high yield.

For unsaturated polymers of structure (I or IV), the di-(p-nitrophenyl)esters of unsaturated dicarboxylic acid can be synthesized from p-nitrophenol and unsaturated dicarboxylic acid chloride, e.g., by dissolving triethylamine and p-nitrophenol in acetone and adding unsaturated dicarboxylic acid chloride dropwise with stirring at −78° C. and pouring into water to precipitate product. Suitable acid chlorides included fumaric, maleic, mesaconic, citraconic, glutaconic, itaconic, ethenyl-butane dioic and 2-propenyl-butanedioic acid chlorides.

Suitable therapeutic diol compounds that can be used to prepare bis(α-amino acid)diesters of therapeutic diol monomers, or active di-ester of therapeutic di-acid monomers, for introduction into the invention epoxy-containing PEA polymer compositions include naturally occurring therapeutic diols, such as 17-β-estradiol, a natural and endogenous hormone, useful in preventing restenosis and tumor growth (Yang, N. N., et al. Identification of an estrogen response element activated by metabolites of 17-β-estradiol and raloxifene. Science (1996) 273, 1222-1225; Parangi, S., et al., Inhibition of angiogenesis and breast cancer in mice by the microtubule inhibitors 2-methoxyestradiol and taxol, Cancer Res. (1997) 57, 81-86; and Fotsis, T., et al., The endogenous oestrogen metabolite 2-methoxyoestradiol inhibits angiogenesis and suppresses tumor growth. Nature (1994) 368, 237-239). The safety profiles of such endogenously occurring therapeutic diol molecules are believed to be superior to those of synthetic and/or non-endogenous molecules having a similar utility, such as sirolimus.

Incorporation of a therapeutic diol into the backbone of a PEA polymer can be accomplished, for example, using active steroid hormone 17-β-estradiol containing mixed hydroxyls—secondary and phenolic. When the PEA polymer is used to fabricate particles and the particles are implanted into a patient, for example, following percutaneous transluminal coronary angioplasty (PTCA), 17-β-estradiol released from the particles in vivo can help to prevent post-implant restenosis in the patient. 17-β-estradiol, however, is only one example of a diol with therapeutic properties that can be incorporated in the backbone of a PEA polymer in accordance with the invention. In one aspect, any bioactive steroid-diol containing primary, secondary or phenolic hydroxyls can be used for this purpose. Many steroid esters that can be made from bioactive steroid diols for use in the invention are disclosed in European application EP 0127 829 A2.

Due to the versatility of the epoxy-containing PEA polymers used in the invention compositions, the amount of the therapeutic diol or di-acid incorporated in the polymer backbone can be controlled by varying the proportions of the building blocks of the polymer. For example, depending on the composition of the PEA, loading of up to 45% w/w of 17-β-estradiol can be achieved. Three different regular, linear PEAs with various loading ratios of 17-β-estradiol are illustrated in Scheme 1 below: 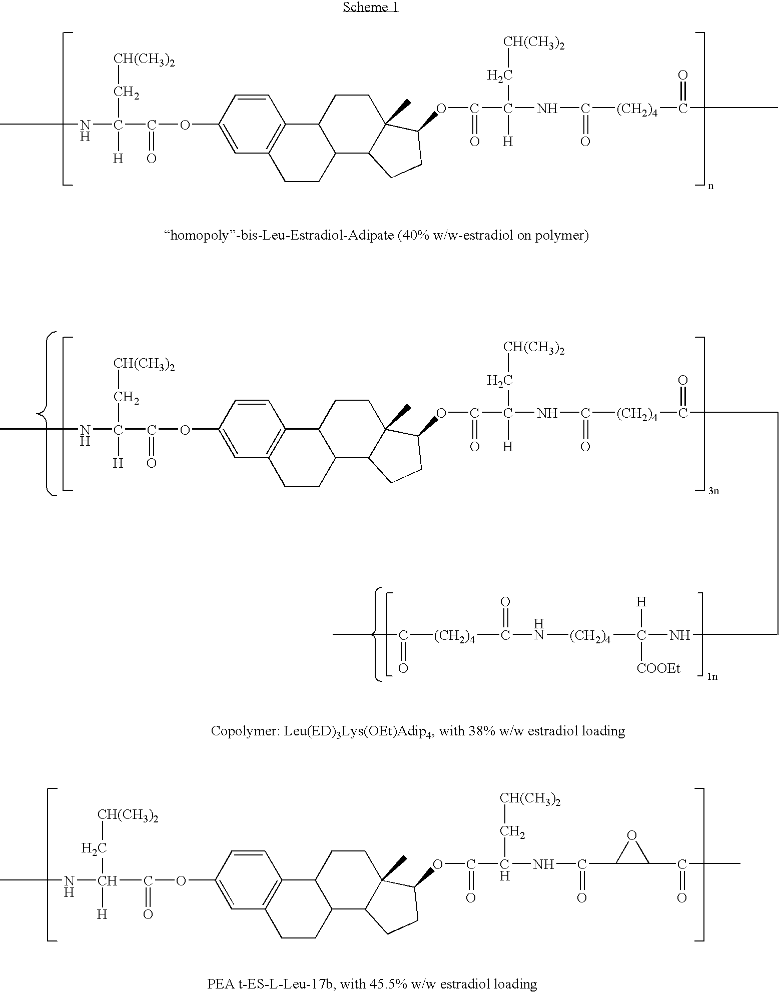
Similarly, the loading of the therapeutic diol into the polymer can be varied by varying the amount of two or more building blocks of the polymer.

In addition, synthetic steroid based diols based on testosterone or cholesterol, such as 4-androstene-3,17 diol(4-Androstenediol), 5-androstene-3,17 diol(5-Androstenediol), 19-nor5-androstene-3,17 diol(19-Norandrostenediol) are suitable for incorporation into the backbone of PEA polymers according to this invention. Moreover, therapeutic diol compounds suitable for use in preparation of the invention epoxy-containing polymer compositions include, for example, amikacin; amphotericin B; apicycline; apramycin; arbekacin; azidamfenicol; bambermycin(s); butirosin; carbomycin; cefpiramide; chloramphenicol; chlortetracycline; clindamycin; clomocycline; demeclocycline; diathymosulfone; dibekacin, dihydrostreptomycin; dirithromycin; doxycycline; erythromycin; fortimicin(s); gentamycin(s); glucosulfone solasulfone; guamecycline; isepamicin; josamycin; kanamycin(s); leucomycin(s); lincomycin; lucensomycin; lymecycline; meclocycline; methacycline; micronomycin; midecamycin(s); minocycline; mupirocin; natamycin; neomycin; netilmicin; oleandomycin; oxytetracycline; paromycin; pipacycline; podophyllinic acid 2-ethylhydrazine; primycin; ribostamycin; rifamide; rifampin; rafamycin SV; rifapentine; rifaximin; ristocetin; rokitamycin; rolitetracycline; rasaramycin; roxithromycin; sancycline; sisomicin; spectinomycin; spiramycin; streptomycin; teicoplanin; tetracycline; thiamphenicol; theiostrepton; tobramycin; trospectomycin; tuberactinomycin; vancomycin; candicidin(s); chlorphenesin; dermostatin(s); filipin; fungichromin; kanamycin(s); leucomycins(s); lincomycin; lvcensomycin; lymecycline; meclocycline; methacycline; micronomycin; midecamycin(s); minocycline; mupirocin; natamycin; neomycin; netilmicin; oleandomycin; oxytetracycline; paramomycin; pipacycline; podophyllinic acid 2-ethylhydrazine; priycin; ribostamydin; rifamide; rifampin; rifamycin SV; rifapentine; rifaximin; ristocetin; rokitamycin; rolitetracycline; rosaramycin; roxithromycin; sancycline; sisomicin; spectinomycin; spiramycin; strepton; otbramycin; trospectomycin; tuberactinomycin; vancomycin; candicidin(s); chlorphenesin; dermostatin(s); filipin; fungichromin; meparticin; mystatin; oligomycin(s); erimycinA; tubercidin; 6-azauridine; aclacinomycin(s); ancitabine; anthramycin; azacitadine; bleomycin(s) carubicin; carzinophillin A; chlorozotocin; chromomcin(s); doxifluridine; enocitabine; epirubicin; gemcitabine; mannomustine; menogaril; atorvasi pravastatin; clarithromycin; leuproline; paclitaxel; mitobronitol; mitolactol; mopidamol; nogalamycin; olivomycin(s); peplomycin; pirarubicin; prednimustine; puromycin; ranimustine; tubercidin; vinesine; zorubicin; coumetarol; dicoumarol; ethyl biscoumacetate; ethylidine dicoumarol; iloprost; taprostene; tioclomarol; amiprilose; romurtide; sirolimus (rapamycin); tacrolimus; salicyl alcohol; bromosaligenin; ditazol; fepradinol; gentisic acid; glucamethacin; olsalazine; S-adenosylmethionine; azithromycin; salmeterol; budesonide; albuteal; indinavir; fluvastatin; streptozocin; doxorubicin; daunorubicin; plicamycin; idarubicin; pentostatin; metoxantrone; cytarabine; fludarabine phosphate; floxuridine; cladriine; capecitabien; docetaxel; etoposide; topotecan; vinblastine; teniposide, and the like. The therapeutic diol can be selected to be either a saturated or an unsaturated diol.

Suitable naturally occurring and synthetic therapeutic di-acids that can be used to prepare an amide linkage in the PEA polymer compositions of the invention include, for example, bambermycin(s); benazepril; carbenicillin; carzinophillin A; cefixime; cefininox cefpimizole; cefodizime; cefonicid; ceforanide; cefotetan; ceftazidime; ceftibuten; cephalosporin C; cilastatin; denopterin; edatrexate; enalapril; lisinopril; methotrexate; moxalactam; nifedipine; olsalazine; penicillin N; ramipril; quinacillin; quinapril; temocillin; ticarcillin; Tomudex® (N-[[5-[[(1,4-Dihydro-2-methyl-4-oxo-6-quinazolinyl)methyl]methylamino]-2-thienyl]carbonyl]-L-glutamic acid), and the like. The safety profile of naturally occurring therapeutic di-acids is believed to surpass that of synthetic therapeutic di-acids. The therapeutic di-acid can be either a saturated or an unsaturated di-acid.

The chemical and therapeutic properties of the above described therapeutic diols and di-acids as tumor inhibitors, cytotoxic antimetabolites, antibiotics, and the like, are well known in the art and detailed descriptions thereof can be found, for example, in the 13th Edition of The Merck Index (Whitehouse Station, N.J., USA).

The di-aryl sulfonic acid salts of bis(α-amino acid)-diesters of saturated and unsaturated diols can be prepared by admixing α-amino acid, aryl sulfonic acid (e.g., p-toluene sulfonic acid monohydrate) and saturated or unsaturated diol in toluene, heating to reflux temperature, until water evolution is minimal, then cooling. The unsaturated diols include, for example, 2-butene-1,4-diol and 1,18-octadec-9-en-diol.

Saturated di-(p-nitrophenyl) esters of dicarboxylic acid and saturated di-p-toluene sulfonic acid salts of bis(α-amino acid)-alkylene-diesters can be prepared as described in U.S. Pat. No. 6,503,538 B1.

Although the invention expoxy-containing PEA polymer compositions are poly(ester amides) (PEAs) made by polycondensation of components as described above, in the present invention, the components can include a di-p-toluenesulfonic acid salt of bis(α-amino acid)-1,4:3,6-dianhydrosorbitol diester; a di-p-toluenesulfonic acid salt of bis(α-amino acid)-aliphatic α,ω-diol diester and a di-(p-nitrophenyl)ester of at least one aliphatic epoxy-diacid or diepoxy-diacid. The di-(p-nitrophenyl)esters of dicarboxylic acids are used because the p-nitrophenyl ester is a very good leaving group that can promote the condensation reaction to move to the right of the reaction equation so the polymer product is obtained in high yield. In addition, the di-(p-nitrophenyl)esters are stable throughout workup and can be handled and dried in open atmosphere.

In unsaturated PEA, the following hold: Aminoxyl radical e.g., 4-amino TEMPO can be attached as described in Example 6 herein. Optionally, bioactive agents, as described herein, can be attached via a double bond functionality, preferably one that does not occur in a residue of a bioactive agent in the polymer backbone. Hydrophilicity, if desired, can be imparted by bonding to poly(ethylene glycol)diacrylate.

The alkylene di-acid-containing PEA polymers described herein have weight average molecular weights ranging from 15,000 to 600,000 Daltons; these polymers and copolymers typically have inherent viscosities at 25° C., determined by standard viscosimetric methods, ranging from 0.25 to 2.0, preferably ranging from 0.4 to 1.7.

The molecular weights and polydispersities herein are determined by gel permeation chromatography (GPC) using polystyrene standards. More particularly, number and weight average molecular weights (Mₙ and M_{w}) are determined, for example, using a Model 510 gel permeation chromatographer (Water Associates, Inc., Milford, Mass.) equipped with a high-pressure liquid chromatographic pump, a Waters 486 UV detector and a Waters 2410 differential refractive index detector. Solution of 0.1% LiCl in N,N-dimethylacetamide (DMAc) is used as the eluent (1.0 mL/min). The polystyrene (PS) standards, with narrow molecular weight distribution, were used for calibration of GPC curves.

The aliphatic epoxy di-acid-containing PEA polymers described herein can be fabricated in a variety of molecular weights and a variety of relative proportions of the two bis(α-amino acid)-diester containing units and optional L-lysine based monomer. The appropriate molecular weight for a particular use is readily determined by one of skill in the art based on the guidelines contained herein and the thermo-mechanical properties disclosed. Thus, e.g., a suitable molecular weight will be on the order of about 15,000 to about 600,000 Daltons, for example about 15,000 to about 300,000, or about 15,000 to about 100,000.

The invention epoxy-containing PEA polymers useful in the invention compositions, and the biodegradable particles or medical devices containing the compositions are enzymatically biodegradable, biodegrading by enzymatic action at the surface rather than by bulk degradation. Therefore, the polymers, for example particles thereof, facilitate in vivo release of a bioactive agent dispersed in the polymer from the surface at a controlled release rate, which is specific and constant over a prolonged period, depending upon the structure of the polymer and the shape of the surface. Additionally, since PEA polymers break down in vivo via enzymes without production of adverse side products, the polymers in the invention compositions and medical devices, such as those that produce biological α-amino acids upon break down, are substantially non-inflammatory.

For convenience the following standard designations of the residues of building blocks of macromolecules described herein are used:

Adipic acid, R¹═(CH₂)₄—<<4>>

Sebacic acid, R¹═(CH₂)₈,—<<8>>

trans-Epoxy succinic acid—<<t-ES>>

cis-Epoxy succinic acid—<<c-ES>>

bis-(L-phenylalanine)-1,6-hexylene diester—<<L-Phe-6>>

bis-(L-leucine)-1,6-hexylene diester—<<L-Leu-6>>

The mole portion of each repeating unit in a co-polymer is designated by subscripts. For example, 8-L-Phe-6 designates homo-poly(ester amide) based on sebacic acid and bis-(L-phenylalanine)-1,6-hexylene diester; t-ES-L-Leu-6 designates homo-poly(ester amide) based on trans-epoxy succinic acid and bis-(L-leucine)-1,6-hexylene diester; [8-L-Phe-6]_{0.6}-[c-ES-L-Phe-6]_{0.4} designates co-poly(ester amide) containing 60 mol. % of repeating units 8-L-Phe-6 and 40 mol. % of repeating units c-ES-L-Phe-6 (polymer examples see or scheme 1).

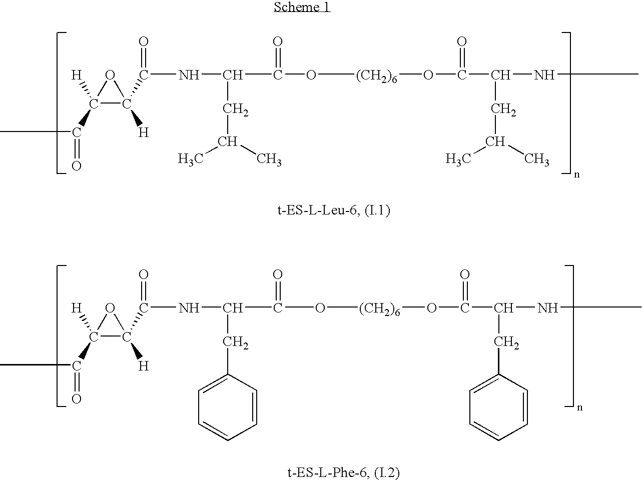

To test versatility of incorporating variable quantities of epoxy-groups in the invention polymers, various co-poly(ester amide)s were fabricated by polycondensation using di-p-nitrophenyl adipate or di-p-nitrophenyl sebacate and the epoxy-containing bis-electrophilic monomers. This approach, apart from providing polymers with the beneficial properties noted above, allows retention of the valuable properties that have already been established of PEAs that do not contain epoxy functionalities (G. Markosishvili et al. (2002) Intern J. Dermatology 41:453 and S. H. Lee, et al. (2002) Coronary Artery Disease, 13 (4):237-241), while providing substantial modification of these properties and, thereby, expanding the scope of potential uses for the polymer.

In contrast to the trans-isomer, which led to the synthesis of high-molecular-weight film-forming homo-polymers and copolymers having reduced viscosity (η_{red}) of up to 0.5-0.6 dL/g, see Table. 2.1), the cis-isomer under the conditions of solution active polycondensation led to the synthesis of homo-polymers with low viscosity (η_{red} under 0.1 dL/g, see Table. 2.1). Copolymers formed using the cis isomer were characterized by slightly higher viscosity (η_{red} up to 0.22 dL/g) and revealed film-forming properties.

The low viscosity characteristics of the polymers based on cis-isomers derived from maleic acid may result from the following factors:

1. Chain termination due to the formation of five-membered epoxy-succinimide cycles in the course of polycondensation;

2. The hydrolysis of vicinal (i.e. in 1,2-position) amide groups owing to the intra-molecular catalysis well known from literature (the invention epoxy-containing PEAs were completely separated in water due to solubility in virtually in all organic solvents, which were used in this case as precipitants).

3. Peculiarities of hydrodynamic parameters of macro-chains in derivatives of cis-epoxy succinic acid, especially in case of copolymers revealing film-forming properties.

Molecular-weight characteristics of these illustrative epoxy-polymers (GPC in DMF/LiBr 0.1%) were as follows:

t-ES-L-Leu-6: M_{w}=66,960, Mₙ=41,120, M_{w}/Mₙ=1.63

t-ES-L-Phe-6: M_{w}=59,100, Mₙ=30,300, M_{w}/Mₙ=1.95

The most probable cause of low viscosity and higher M_{w} in the polymers based on di-p-nitrophenyl-cis-epoxy succinate (V.5) is mutual intramolecular catalysis of hydrolysis of 1,2-amide groups. A prolonged (3-4 weeks) contact with water is unavoidable to purify the polymers from low-molecular-weight by-products of polycondensation, which are mostly sparely soluble in water p-nitrophenol, making this cause especially probable. The solvent DMA and p-toluenesulfonic acid salt of triethylamine are completely removed after the first day of the treatment with water.

TABLE 1.1
Epoxy-poly(ester amide)s obtained by active polycondensation

| | | Yield, | η_{red}*⁾, | Solubility**⁾ | | |
|---|---|---|---|---|---|---|
| No | Polymer | [%] | [dL/g] | Acetone | Chloroform | DMF |
| 1 | t-ES-L-Phe-6 | 89.0 | 0.58 | ± | + | + |
| 2 | t-ES-L-Leu-6 | 90.7 | 0.53 | + | + | + |
| 3 | [4-L-Phe-4]_{0.6}- | 95.0 | 0.20 | ± | + | + |
| | [t-ES-L-Phe-4]_{0.4} | | | | | |
| 4 | [8-L-Phe-6]_{0.6}- | 94.0 | 0.23 | ± | + | + |
| | [t-ES-L-Phe-6]_{0.4} | | | | | |
| 5 | [8-L-Leu-6]_{0.6}- | 93.6 | 0.48 | + | + | + |
| | [t-ES-L-Leu-6]_{0.4} | | | | | |
| 6 | c-ES-L-Phe-6 | 85.8 | 0.09 | ± | + | + |
| 7 | c-ES-L-Leu-6 | 79.8 | 0.08 | + | + | + |
| 8 | [8-L-Phe-6]_{0.6}- | 84.7 | 0.22 | ± | + | + |
| | [c-ES-L-Phe-6]_{0.4} | | | | | |
| 9 | [8-L-Leu-6]_{0.6}- | 85.6 | 0.21 | + | + | + |
| | [c-ES-L-Leu-6]_{0.4} | | | | | |

*⁾Determined in DMF at c = 0.5 g/dL and t = 25° C.
**⁾<<+>> - soluble, <<±>> - partially soluble.

IR-studies (see Example 3 below) have confirmed that the epoxy-containing PEAs obtained are thermo-reactive materials, which are of interest for preparing biodegradable polymeric networks with enhanced mechanical characteristics. The invention polymers can also be used as additives to improve mechanical properties of other biodegradable, biocompatible polymers, such as other PEAs, poly(ester ureas) (PEUs) and poly(ester urethanes) (PEURs).

Photochemical curing. The invention epoxy-containing PEAs also undergo photochemical cross-linking (curing) after UV-irradiation as was confirmed by loss of solubility in organic solvents in which the polymers are soluble before irradiation, such as chloroform, ethanol, DMF, and the like. The photochemical reaction proceeds with either high intensity broad-band UV exposure or in the presence of specific catalysts (such as, cationic photo initiators, e.g., onium salts of sulfur or phosphorous organic compounds) normally used for photochemical transformations of regular epoxides.

Chemical curing: As noted above, the invention epoxy-containing PEAs interact with fatty amines under mild conditions. This reaction has been used for chemical curing of these polymers under mild conditions (i.e., room temperature). For this method of chemical curing, 0.1 g of epoxy-polymer—t-ES-L-Leu-6 (I.1) and 1,6-hexamethylene diamine (as fatty diamine, 10% w/w) was dissolved in 2 mL of chloroform, cast onto a smooth hydrophobic surface and left overnight under atmospheric conditions while chloroform was evaporated up to dryness. An elastic film insoluble in chloroform (swelled only) was obtained.

Various aliphatic epoxy-di-acids, derived from non-toxic aliphatic di-acids, such as succinic or adipic acid, have been implemented in this study as starting compounds. For example trans-and cis-epoxysuccinic acids are commercially available. In this study these acids have been prepared by epoxydation reaction of fumaric or maleic acid, respectively. The di-acids so formed were further converted to their p-nitrophenyl ester and used as an activated monomer in active polycondensation to form the invention epoxy-containing PEAs.

Active diesters (Compounds V.1-5) that have been synthesized for use in synthesis of the invention epoxy-containing PEAs are summarized in Table 1.1 and Table 1.2:

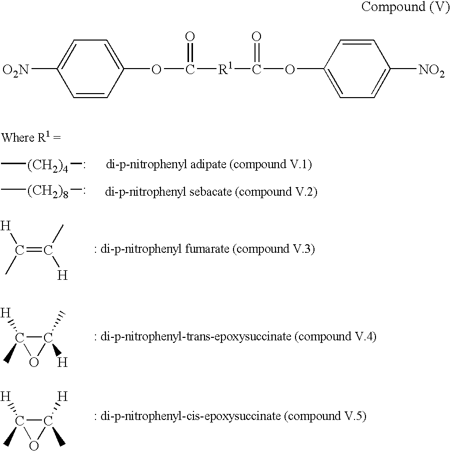

Syntheses of aliphatic saturated and unsaturated di-esters were conducted as described previously (Guo K. et al. J. Polym. Sci: Part A: Polymer Chemistry (2005) 43:1463-1477 and therein cited references).

Unsaturated active di-ester, di-p-nitrophenyl fumarate (R¹═—HC═CH—, compound V.3) was also obtained in 85-90% yield as an intermediate product for synthesis of the epoxy-monomer using a second strategic approach. For example, such sample compound was synthesized in a medium of organic solvent (acetone) by reaction of fumaryl chloride with p-nitrophenol in the presence of tertiary amine. After re-crystallization from acetone, the product melting point (m.p.) was 234-235° C. (literature m.p. (Guo et al., supra)=238° C., from acetonitrile).

Synthesis of epoxy-acids from their unsaturated precursor di-acids (fumaric and maleic acids) was achieved by using hydrogen peroxide. During epoxydation the reaction conditions were selected, as is known in the art and as illustrated in the Examples herein, such that the consequent cleavage of the oxyrane ring via hydrolysis was minimized.

In certain embodiments, a bioactive agent can be covalently bound to the biodegradable polymers via a wide variety of suitable functional groups. For example, when the biodegradable polymer is a polyester, the carboxylic group chain end can be used to react with a complimentary moiety on the bioactive agent, such as hydroxy, amino, thio, and the like. A wide variety of suitable reagents and reaction conditions are disclosed, e.g., in March's Advanced Organic Chemistry, Reactions, Mechanisms, and Structure, Fifth Edition, (2001); and Comprehensive Organic Transformations, Second Edition, Larock (1999).

In other embodiments, a bioactive agent can be dispersed into the polymer by “loading” onto the polymer without formation of a chemical bond or the bioactive agent can be linked to any free functional group in the polymers, such as an amine, hydroxyl (alcohol), or thiol, and the like, to form a direct linkage. Such a linkage can be formed from suitably functionalized starting materials using synthetic procedures that are known in the art.

For example, a polymer of the present invention can be linked to the bioactive agent via a carboxyl group (e.g., COOH) of the polymer. Specifically, a compound of structures (I and IV) can react with an amino functional group of a bioactive agent or a hydroxyl functional group of a bioactive agent to provide a biodegradable, biocompatible polymer having the bioactive agent attached via an amide linkage or ester linkage, respectively. In another embodiment, the carboxyl group of the polymer can be transformed into an acyl halide, acyl anhydride/“mixed” anhydride, or active ester.

Alternatively, the bioactive agent may be attached to the polymer via a linker. Indeed, to improve surface hydrophobicity of the biodegradable polymer, to improve accessibility of the biodegradable polymer towards enzyme activation, and to improve the release profile of the biodegradable polymer, a linker may be utilized to indirectly attach the bioactive agent to the biodegradable polymer. In certain embodiments, the linker compounds include poly(ethylene glycol) having a molecular weight (Mw) of about 44 to about 10,000, preferably 44 to 2000; amino acids, such as serine; polypeptides with repeat units from 1 to 100; and any other suitable low molecular weight polymers. The linker typically separates the bioactive agent from the polymer by about 5 angstroms up to about 200 angstroms.

In still further embodiments, the linker is a divalent radical of formula W-A-Q, wherein A is (C₁-C₂₄)alkyl, (C₂-C₂₄)alkenyl, (C₂-C₂₄)alkynyl, (C₂-C₂₀)alkyloxy, (C₃-C₈)cycloalkyl, or (C₆-C₁₀)aryl, and W and Q are each independently —N(R)C(═O)—, —C(═O)N(R)—, —OC(═O)—, —C(═O)O, —O—, —S—, —S(O), —S(O)₂—, —S—S—, —N(R)—, —C(═O)—, wherein each R is independently H or (C₁-C₆)alkyl. In still further embodiments, the linker is a divalent radical of formula W-A-Q, wherein A is (C₁-C₂₄)alkyl, (C₂-C₂₄)alkenyl, (C₂-C₂₄)alkynyl, (C₂-C₂₀)alkyloxy, (C₃-C₈) cycloalkyl, or (C₆-C₁₀)aryl, and W and Q are each independently —N(R)C(═O)—, —C(═O)N(R)—, —OC(═O)—, —C(═O)O, —O—, —S—, —S(O), —S(O)₂—, —S—S—, —N(R)—, —C(═O)—, wherein each R is independently H or (C₁-C₆) alkyl.

As used herein, the term “alkyl”, as applied to the linkers described herein, refers to a straight or branched chain hydrocarbon group including methyl, ethyl, n-propyl, isopropyl, n-butyl, isobutyl, tert-butyl, n-hexyl, and the like.

As used herein, “alkenyl”, as applied to the linkers described herein, refers to straight or branched chain hydrocarbon groups having one or more carbon-carbon double bonds.

As used herein, “alkynyl”, as applied to the linkers described herein, refers to straight or branched chain hydrocarbon groups having at least one carbon-carbon triple bond.

As used herein, “aryl”, as applied to the linkers described herein, refers to aromatic groups having in the range of 6 up to 14 carbon atoms.

In certain embodiments, the linker may be a polypeptide having from about 2 up to about 25 amino acids. Suitable peptides contemplated for use include poly-L-lysine, poly-L-glutamic acid, poly-L-aspartic acid, poly-L-histidine, poly-L-ornithine, poly-L-threonine, poly-L-tyrosine, poly-L-leucine, poly-L-lysine-L-phenylalanine, poly-L-arginine, poly-L-lysine-L-tyrosine, and the like.

The linker can be attached first to the polymer or to the bioactive agent. During synthesis of polymers having bioactive agents indirectly attached via a linker, the linker can be either in unprotected form or protected from, using a variety of protecting groups well known to those skilled in the art.

In the case of a protected linker, the unprotected end of the linker can first be attached to the polymer or the bioactive agent. The protecting group can then be de-protected using Pd/H₂ hydrogenolysis for saturated polymers, mild acid or base hydrolysis for unsaturated polymers, or any other common de-protection method that is known in the art. The de-protected linker can then be attached to the bioactive agent. Poly(ethylene glycol) can also be employed as the linker between polymer and bioactive agent.

Illustrations of syntheses of polymer compositions according to the invention are found in the Examples herein.

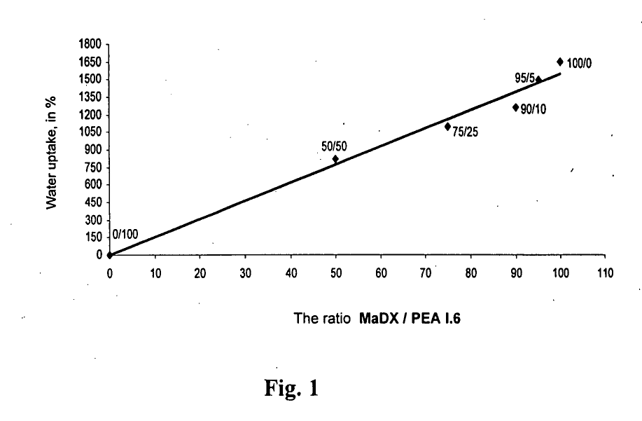

In another embodiment, the invention provides biodegradable three-dimensional hybrid networks from reactive derivatives of both epoxy-containing PEAs and polysaccharides. Examples of reactivated derivative of invention epoxy-containing PEAs are those that contain (meth)acryloyl moieties, such as unsaturated compound PEA I.6. A mixture of the two components is cast onto a substrate in an appropriate solvent, such as DMA, dried, and heated to a temperature and for a time sufficient to cause formation of a three-dimensional hydrogel, for example to a temperature of about 80° C. to about 120° C. for a period of from about 6 hours to about 10 hours. The reactive derivative of an invention epoxy-containing PEA is one modified to contain acrilic pending chain, such as Compound I.6 described in Example 3 herein. For example the w/w ratio of dextran to reactivate PEA, can be in the range from about 95:5, about 50:50, for example a w/w ratio of about 90:10 or about 75:25. Preferably the mixture of dextran and activated epoxy-containing PEA is dissolved in DMF (1 g in 10 mL) and cast onto a substrate to dry the solvent, forming a film thereon prior to heating. A free-radical initiator, such as benzoyl peroxide can be added (1% of the mixture of MaDX+I.6), but is not needed for formation of the hydrogel. Films obtained after heating can imbibe water, changing the swelling index by about 300% to about 900% depending on the ratio of the polysaccharide to the polymer (FIG. 1).

While the optional bioactive agent(s) can be dispersed within the polymer matrix without chemical linkage to the polymer carrier, it is also contemplated that one or more bioactive agents or covering molecules can be covalently bound to the biodegradable polymers via a wide variety of suitable functional groups. For example, a free carboxyl group can be used to react with a complimentary moiety on a bioactive agent or covering molecule, such as a hydroxy, amino, or thio group, and the like. A wide variety of suitable reagents and reaction conditions are disclosed, e.g., in March's Advanced Organic Chemistry, Reactions, Mechanisms, and Structure, Fifth Edition, (2001); and Comprehensive Organic Transformations, Second Edition, Larock (1999).

In other embodiments, one or more bioactive agent can be linked to any of the polymers of structures (I and IV) through an amide, ester, ether, amino, thioether, sulfinyl, sulfonyl, or disulfide linkage. Such a linkage can be formed from suitably functionalized starting materials using synthetic procedures that are known in the art.

For example, in one embodiment a polymer can be linked to a bioactive agent or adjuvant via a free carboxyl group (e.g., COOH) of the polymer. Specifically, a compound of structures (I) and (IV) can react with an amino functional group or a hydroxyl functional group of a bioactive agent to provide a biodegradable polymer having the bioactive agent attached via an amide linkage or ester linkage, respectively. In another embodiment, the carboxyl group of the polymer can be benzylated or transformed into an acyl halide, acyl anhydride/“mixed” anhydride, or active ester. In other embodiments, the free —NH₂ ends of the polymer molecule can be acylated to assure that the bioactive agent will attach only via a carboxyl group of the polymer and not to the free ends of the polymer.

The invention epoxy-containing PEA polymer compositions can be formulated into particles to provide a variety of properties. The particles can have a variety of sizes and structures suitable to meet differing therapeutic goals and routes of administration using methods described in full in co-pending U.S. application Ser. No. 11/344,689, filed Jan. 31, 2006.)

Water soluble covering molecule(s), such as poly(ethylene glycol) (PEG); phosphatidylcholine (PC); glycosaminoglycans including heparin; polysaccharides including chitosan, alginates and polysialic acid; poly(ionizable or polar amino acids) including polyserine, polyglutamic acid, polyaspartic acid, polylysine and polyarginine; as described herein, and targeting molecules, such as antibodies, antigens and ligands, are bioactive agents that can also be conjugated to the polymer on the exterior of particles or medical devices formed from the invention polymer compositions after production to block active sites thereon not occupied by a bioactive agent or to target delivery of the particles to a specific body site as is known in the art. The molecular weights of PEG molecules on a single particle can be substantially any molecular weight in the range from about 200 to about 200,000, so that the molecular weights of the various PEG molecules attached to the particle can be varied.

Alternatively, a bioactive agent or covering molecule can be attached to the polymer via a linker molecule. Indeed, to improve surface hydrophobicity of the biodegradable polymer, to improve accessibility of the biodegradable polymer towards enzyme activation, and to improve the release profile of the bioactive agents from the biodegradable polymer, a linker may be utilized to indirectly attach a bioactive agent to the biodegradable polymer. In certain embodiments, the linker compounds include poly(ethylene glycol) having a molecular weight (Mw) of about 44 to about 10,000, preferably 44 to 2000; amino acids, such as serine; polypeptides with repeat number from 1 to 100; and any other suitable low molecular weight polymers. The linker typically separates the bioactive agent from the polymer by about 5 angstroms up to about 200 angstroms.

In still further embodiments, the linker is a divalent radical of formula W-A-Q, wherein A is (C₁-C₂₄)alkyl, (C₂-C₂₄)alkenyl, (C₂-C₂₄)alkynyl, (C₂-C₂₀)alkyloxy, (C₃-C₈)cycloalkyl, or (C₆-C₁₀)aryl, and W and Q are each independently —N(R)C(═O)—, —C(═O)N(R)—, —OC(═O)—, —C(═O)O, —O—, —S—, —S(O), —S(O)₂—, —S—S—, —N(R)—, —C(═O)—, wherein each R is independently H or (C₁-C₆)alkyl.

As used to describe the above linkers, the term “alkyl” refers to a straight or branched chain hydrocarbon group including methyl, ethyl, n-propyl, isopropyl, n-butyl, isobutyl, tert-butyl, n-hexyl, and the like.

As used to describe the above linkers, “alkenyl” refers to straight or branched chain hydrocarbyl groups having one or more carbon-carbon double bonds.

As used to describe the above linkers, “alkynyl” refers to straight or branched chain hydrocarbyl groups having at least one carbon-carbon triple bond.

As used to describe the above linkers, “aryl” refers to aromatic groups having in the range of 6 up to 14 carbon atoms.

In certain embodiments, the linker may be a polypeptide having from about 2 up to about 25 amino acids. Suitable peptides contemplated for use include poly-L-glycine, poly-L-lysine, poly-L-glutamic acid, poly-L-aspartic acid, poly-L-histidine, poly-L-ornithine, poly-L-serine, poly-L-threonine, poly-L-tyrosine, poly-L-leucine, poly-L-lysine-L-phenylalanine, poly-L-arginine, poly-L-lysine-L-tyrosine, and the like.

In one embodiment, a bioactive agent can covalently crosslink the polymer, i.e. the bioactive agent is bound to more than one polymer molecule, to form an intermolecular bridge. This covalent crosslinking can be done with or without a linker containing a bioactive agent.

A bioactive agent molecule can also be incorporated into an intramolecular bridge by covalent attachment between two sites on the same polymer molecule.

A linear polymer polypeptide conjugate is made by protecting the potential nucleophiles on the polypeptide backbone and leaving only one reactive group to be bound to the polymer or polymer linker construct. Deprotection is performed according to methods well known in the art for deprotection of peptides (Boc and Fmoc chemistry for example).

In one embodiment of the present invention, a bioactive agent is a polypeptide presented as a retro-inverso or partial retro-inverso peptide.

In other embodiments, a bioactive agent may be mixed with a photocrosslinkable version of the polymer in a matrix, and, after crosslinking, the material is dispersed (ground) to form particles having an average diameter in the range from about 0.1 to about 10 μm.

The linker can be attached first to the polymer or to the bioactive agent or covering molecule. During synthesis, the linker can be either in unprotected form or protected from, using a variety of protecting groups well known to those skilled in the art. In the case of a protected linker, the unprotected end of the linker can first be attached to the polymer or the bioactive agent or covering molecule. The protecting group can then be de-protected using Pd/H₂ hydrogenation for saturated polymer backbones, mild acid or base hydrolysis for unsaturated polymers, or any other common de-protection method that is known in the art. The de-protected linker can then be attached to the bioactive agent or covering molecule, or to the polymer.

An exemplary conjugate synthesis performed on a biodegradable polymer according to the invention (wherein the molecule to be attached to the polymer is an amino substituted aminoxyl N-oxide radical) is set forth as follows. A biodegradable polymer herein can be reacted with an aminoxyl radical containing compound, e.g., 4-amino-2,2,6,6-tetramethylpiperidine-1-oxy, in the presence of N,N′-carbonyl diimidazole or suitable carbodiimide, to replace the hydroxyl moiety in the carboxyl group, either on the pendant carboxylic acids of the PEAs or UPEAs, or at the chain end of a polyester as described, with an amide linkage to the aminoxyl (N-oxide) radical containing group. The amino moiety covalently bonds to the carbon of the carbonyl residue such that an amide bond is formed. The N,N′-carbonyldiimidazole or suitable carbodiimide converts the hydroxyl moiety in the carboxyl group at the chain end of the polyester into an intermediate activated moiety which will react with the amino group of the aminoxyl (N oxide) radical compound, e.g., the amine at position 4 of 4-amino-2,2,6,6-tetramethylpiperidine-1-oxy. The aminoxyl reactant is typically used in a mole ratio of reactant to polyester ranging from 1:1 to 100:1. The mole ratio of N,N′-carbonyldiimidazole or carbodiimide to aminoxyl is preferably about 1:1.

A typical reaction is as follows. A polyester is dissolved in a reaction solvent and reaction is readily carried out at the temperature utilized for the dissolving. The reaction solvent may be any in which the polyester will dissolve; this information is normally available from the manufacturer of the polyester. When the polyester is a polyglycolic acid or a poly(glycolide-L-lactide) (having a monomer mole ratio of glycolic acid to L-lactic acid greater than 50:50), highly refined (99.9+% pure) dimethyl sulfoxide at 115° C. to 130° C. or DMSO at room temperature suitably dissolves the polyester. When the polyester is a poly-L-lactic acid, a poly-DL-lactic acid or a poly(glycolide-L-lactide) (having a monomer mole ratio of glycolic acid to L-lactic acid 50:50 or less than 50:50), tetrahydrofuran, dichloromethane (DCM) and chloroform at room temperature to 40˜50° C. suitably dissolve the polyester.

Polymer—Bioactive Agent Linkage

In one embodiment, the polymers used to make the invention epoxy-containing PEA polymer compositions as described herein have one or more bioactive agent directly linked to the polymer. The residues of the polymer can be linked to the residues of the one or more bioactive agents. For example, one residue of the polymer can be directly linked to one residue of a bioactive agent. The polymer and the bioactive agent can each have one open valence. Alternatively, more than one bioactive agent, multiple bioactive agents, or a mixture of bioactive agents having different therapeutic or palliative activity can be directly linked to the polymer. However, since the residue of each bioactive agent can be linked to a corresponding residue of the polymer, the number of residues of the one or more bioactive agents can correspond to the number of open valences on the residue of the polymer.

As used herein, a “residue of a polymer” refers to a radical of a polymer having one or more open valences. Any synthetically feasible atom, atoms, or functional group of the polymer (e.g., on the polymer backbone or pendant group) is substantially retained when the radical is attached to a residue of a bioactive agent. Additionally, any synthetically feasible functional group (e.g., carboxyl) can be created on the polymer (e.g., on the polymer backbone as a pendant group or as chain termini) to provide the open valence, provided bioactivity of the backbone bioactive agent is substantially retained when the radical is attached to a residue of a bioactive agent. Based on the linkage that is desired, those skilled in the art can select suitably functionalized starting materials that can be used to derivatize the PEA polymers used in the present invention using procedures that are known in the art.

As used herein, a “residue of a compound of structural formula (*)” refers to a radical of a compound of polymer formulas (I or IV) as described herein having one or more open valences. Any synthetically feasible atom, atoms, or functional group of the compound (e.g., on the polymer backbone or pendant group) can be removed to provide the open valence, provided bioactivity of the backbone bioactive agent is substantially retained when the radical is attached. Additionally, any synthetically feasible functional group (e.g., carboxyl) can be created on the compound of formulas (I or IV) (e.g., on the polymer backbone or pendant group) to provide the open valence, provided bioactivity of the backbone bioactive agent is substantially retained when the radical is attached to a residue of a bioactive agent. Based on the linkage that is desired, those skilled in the art can select suitably functionalized, starting materials that can be used to derivatize the compound of formulas (I or IV) using procedures that are known in the art.

For example, the residue of a bioactive agent can be linked to the residue of a compound of structural formulas (I and IV) through an amide (e.g., —N(R)C(═O)— or C(═O)N(R)—), ester (e.g., —OC(═O)— or —C(═O)O—, ether (e.g., —O—), amino (e.g., —N(R)—), ketone (e.g., —C(═O)—), thioether (e.g., —S—), sulfinyl (e.g., —S(O)—), sulfonyl (e.g., —S(O)₂—), disulfide (e.g., —S—S—), or a direct (e.g., C—C bond) linkage, wherein each R is independently H or (C₁-C₆)alkyl. Such a linkage can be formed from suitably functionalized starting materials using synthetic procedures that are known in the art. Based on the linkage that is desired, those skilled in the art can select suitably functional starting material to derivatize any residue of a compound of structural formulas (I, IV or V) and thereby conjugate a given residue of a bioactive agent using procedures that are known in the art. The residue of the optional bioactive agent can be linked to any synthetically feasible position on the residue of a compound of structural formulas (I or IV). Additionally, the invention also provides compounds having more than one residue of a bioactive agent directly linked to a compound of structural formulas (I or IV).

The number of bioactive agents that can be linked to the polymer molecule can typically depend upon the molecular weight of the polymer. For example, for a compound of structural formula (I), wherein n is about 5 to about 150, preferably about 5 to about 70, up to about 300 bioactive agent molecules (i.e., residues thereof) can be directly linked to the polymer (i.e., residue thereof) by reacting the bioactive agent with terminal groups of the polymer. On the other hand, for a compound of structural formula (IV) up to an additional 150 bioactive agents can be linked to the polymer by reacting the bioactive agent with the pendant group on the lysine-containing unit. In unsaturated polymers, additional bioactive agents can also be reacted with double (or triple) bonds in the polymer.

The invention epoxy-containing PEA composition, whether used in the form of a polymer depot implant, as particles, or to fabricate a biodegradable medical device, such as a vascular stent, can be covalently attached directly to the bioactive agent; rather than the bioactive agent being dispersed or “loaded” into the polymer without chemical attachment. Any of several methods well known in the art and as described hereinbelow can be used to form the chemical attachment. The amount of bioactive agent is generally approximately 0.1% to about 60% (w/w) bioactive agent to polymer composition, more preferably about 1% to about 25% (w/w) bioactive agent, and even more preferably about 2% to about 20% (w/w) bioactive agent. The percentage of bioactive agent will depend on the desired dose and the condition being treated, as discussed in more detail below.

In addition to serving as a stand-alone delivery system for bioactive agents when directly administered in vivo in the form of implantable particles or an implantable polymer depot, and the like, the invention epoxy-containing PEA compositions can be used in the fabrication of various types of surgical devices. In this embodiment, the invention polymer composition used in fabrication of the medical device is effective for controlled delivery to surrounding tissue of any bioactive agents dispersed in the invention polymer composition, for example, covalently attached to the surface thereof.

Thus in yet another embodiment, the invention epoxy-containing PEA polymer composition has sufficient film-forming and cross-linking characteristics to be used in fabrication of a biodegradable, biocompatible surgical device, including but not limited to internal fixation devices, such as surgical suture, surgical screws, implantable plates, and implantable rods, or vascular stents and dialysis shunts. Any method known in the art for fabrication of a polymer material into a medical device, such as by mold casting, mold cross-linking, and the like, can be used to fabricate a biodegradable surgical device using the invention epoxy-containing PEA compositions, which are cross-linked using any of the methods described herein to form a solid. Such biodegradable, biocompatible medical devices slowly biodegrade, for example over a period of from about two weeks to about six months depending on the degree of cross-linking and the physical dimensions of the device, to create substantially biocompatible breakdown products.

In another embodiment the invention provides methods for delivering a bioactive agent to a subject in need thereof comprising implanting an invention composition at an interior body site so that the composition slowly biodegrades, for example completely. Any bioactive agent dispersed in the polymer will be slowly released during biodegradation to tissue surrounding a site of implantation, for example to promote healing and alleviate pain therein.

In another embodiment, the invention epoxy-containing PEA polymer composition can be fabricated in the form of a biodegradable, biocompatible pad, sheet or wrap of any desired surface area. For example, the polymer can be woven or formed as a thin sheet of randomly oriented fibers by electrospinning to produce nanofibers of the polymer. Such pads, sheets and wraps can be used in a number of types of wound dressings for treatment of a variety of conditions, for example by promoting endogenous healing processes at a wound site. The polymer compositions in the wound dressing biodegrade over time, releasing the bioactive agent to be absorbed into a wound site where it acts intracellularly, either within the cytosol, the nucleus, or both of a target cell, or the bioactive agent can bind to a cell surface receptor molecule to elicit a cellular response without entering the cell. Alternatively, the bioactive agent can be released from the surgical device, such as a vascular stent, having at least one surface partially coated with the invention composition to promote endogenous healing processes at the wound site by contact with the surroundings into which the medical device is implanted. A detailed description of wound dressings, wound healing implants and surgical device coatings made using PEA polymers is found in co-pending U.S. patent application Ser. No. 11/128,903, filed May 12, 2005.

Bioactive agents contemplated for dispersion within the polymers used in the invention epoxy-containing PEA polymer compositions include anti-proliferants, rapamycin and any of its analogs or derivatives, paclitaxel or any of its taxene analogs or derivatives, everolimus, sirolimus, tacrolimus, or any of its -limus named family of drugs, and statins such as simvastatin, atorvastatin, fluvastatin, pravastatin, lovastatin, rosuvastatin, geldanamycins, such as 17AAG (17-allylamino-17-demethoxygeldanamycin); Epothilone D and other epothilones, 17-dimethylaminoethylamino-17-demethoxy-geldanamycin and other polyketide inhibitors of heat shock protein 90 (Hsp90), cilostazol, and the like.

Suitable bioactive agents for dispersion in the invention epoxy-containing PEA polymer compositions and particles made therefrom also can be selected from those that promote endogenous production of a therapeutic natural wound healing agent, such as nitric oxide, which is endogenously produced by endothelial cells. Alternatively the bioactive agents released from the polymers during degradation may be directly active in promoting natural wound healing processes by endothelial cells. These bioactive agents can be any agent that donates, transfers, or releases nitric oxide, elevates endogenous levels of nitric oxide, stimulates endogenous synthesis of nitric oxide, or serves as a substrate for nitric oxide synthase or that inhibits proliferation of smooth muscle cells. Such agents include, for example, aminoxyls, furoxans, nitrosothiols, nitrates and anthocyanins; nucleosides such as adenosine and nucleotides such as adenosine diphosphate (ADP) and adenosine triphosphate (ATP); neurotransmitter/neuromodulators such as acetylcholine and 5-hydroxytryptamine (serotonin/5-HT); histamine and catecholamines such as adrenalin and noradrenalin; lipid molecules such as sphingosine-1-phosphate and lysophosphatidic acid; amino acids such as arginine and lysine; peptides such as the bradykinins, substance P and calcium gene-related peptide (CGRP), and proteins such as insulin, vascular endothelial growth factor (VEGF), and thrombin.

A variety of bioactive agents, coating molecules and ligands for bioactive agents can be attached, for example covalently, to the surface of the polymer particles. Bioactive agents, such as targeting antibodies, polypeptides (e.g., antigens) and drugs can be covalently conjugated to the surface of the polymer particles. In addition, coating molecules, such as polyethylene glycol (PEG) as a ligand for attachment of antibodies or polypeptides or phosphatidylcholine (PC) as a means of blocking attachment sites on the surface of the particles, can be surface-conjugated to the particles to prevent the particles from sticking to non-target biological molecules and surfaces in a subject to which the particles are administered.

For example, small proteinaceous motifs, such as the B domain of bacterial Protein A and the functionally equivalent region of Protein G are known to bind to, and thereby capture, antibody molecules by the Fc region. Such proteinaceous motifs can be attached as bioactive agents to the invention polymers and compositions, especially to the surface of the polymer particles described herein. Such molecules will act, for example, as ligands to attach antibodies for use as targeting ligands or to capture antibodies to hold precursor cells or capture cells out of the blood stream. Therefore, the antibody types that can be attached to polymer coatings using a Protein A or Protein G functional region are those that contain an Fc region. The capture antibodies will in turn bind to and hold precursor cells, such as progenitor cells, near the polymer surface while the precursor cells, which are preferably bathed in a growth medium within the polymer, secrete various factors and interact with other cells of the subject. In addition, one or more bioactive agents dispersed in the polymer particles, such as the bradykinins, may activate the precursor cells.

In addition, bioactive agents for attaching precursor cells or for capturing progenitor endothelial cells (PECs) from a blood stream in a subject to which the polymer compositions are administered are monoclonal antibodies directed against a known precursor cell surface marker. For example, complementary determinants (CDs) that have been reported to decorate the surface of endothelial cells include CD31, CD34, CD102, CD105, CD106, CD109, CDw130, CD141, CD142, CD143, CD144, CDw145, CD146, CD147, and CD166. These cell surface markers can be of varying specificity and the degree of specificity for a particular cell/developmental type/stage is in many cases not fully characterized. In addition, these cell marker molecules against which antibodies have been raised will overlap (in terms of antibody recognition) especially with CDs on cells of the same lineage: monocytes in the case of endothelial cells. Circulating endothelial progenitor cells are some way along the developmental pathway from (bone marrow) monocytes to mature endothelial cells. CDs 106, 142 and 144 have been reported to mark mature endothelial cells with some specificity. CD34 is presently known to be specific for progenitor endothelial cells and therefore is currently preferred for capturing progenitor endothelial cells out of blood in the site into which the polymer particles are implanted for local delivery of the active agents. Examples of such antibodies include single-chain antibodies, chimeric antibodies, monoclonal antibodies, polyclonal antibodies, antibody fragments, Fab fragments, IgA, IgG, IgM, IgD, IgE and humanized antibodies, and active fragments thereof.

The following bioactive agents and small molecule drugs will be particularly effective for dispersion within the invention epoxy-containing PEA polymer compositions. The bioactive agents that are dispersed in the invention epoxy-containing polymer compositions and medical devices made thereof will be selected for their suitable therapeutic or palliative effect in treatment of a wound or disease of interest, or symptoms thereof, or in experiments designed for in vitro testing of such effects in cells or tissue culture, or in vivo.

In one embodiment, the suitable bioactive agents are not limited to, but include, various classes of compounds that facilitate or contribute to wound healing when presented in a time-release fashion. Such bioactive agents include wound-healing cells, including certain precursor cells, which can be protected and delivered by the biodegradable polymer in the invention compositions. Such wound healing cells include, for example, pericytes and endothelial cells, as well as inflammatory healing cells. To recruit such cells to the site of a polymer depot in vivo, the invention epoxy-containing PEA polymer compositions and particles thereof used in the invention and methods of use can include ligands for such cells, such as antibodies and smaller molecule ligands, that specifically bind to “cellular adhesion molecules” (CAMs). Exemplary ligands for wound healing cells include those that specifically bind to Intercellular adhesion molecules (ICAMs), such as ICAM-1 (CD54 antigen); ICAM-2 (CD102 antigen); ICAM-3 (CD50 antigen); ICAM-4 (CD242 antigen); and ICAM-5; Vascular cell adhesion molecules (VCAMs), such as VCAM-1 (CD106 antigen); Neural cell adhesion molecules (NCAMs), such as NCAM-1 (CD56 antigen); or NCAM-2; Platelet endothelial cell adhesion molecules PECAMs, such as PECAM-1 (CD31 antigen); Leukocyte-endothelial cell adhesion molecules (ELAMs), such as LECAM-1; or LECAM-2 (CD62E antigen), and the like.

In another aspect, the suitable bioactive agents include extra cellular matrix proteins, macromolecules that can be dispersed into the polymer particles used in the invention epoxy-containing PEA polymer compositions, e.g., attached either covalently or non-covalently. Examples of useful extra-cellular matrix proteins include, for example, glycosaminoglycans, usually linked to proteins (proteoglycans), and fibrous proteins (e.g., collagen; elastin; fibronectins and laminin). Bio-mimics of extra-cellular proteins can also be used. These are usually non-human, but biocompatible, glycoproteins, such as alginates and chitin derivatives. Wound healing peptides that are specific fragments of such extra-cellular matrix proteins and/or their bio-mimics can also be used.

Proteinaceous growth factors are another category of bioactive agents suitable for dispersion in the invention epoxy-containing PEA polymer compositions and methods of use described herein. Such bioactive agents are effective in promoting wound healing and other disease states as is known in the art, for example, Platelet Derived Growth Factor-BB (PDGF-BB), Tumor Necrosis Factor-alpha (TNF-alpha), Epidermal Growth Factor (EGF), Keratinocyte Growth Factor (KGF), Thymosin B4; and, various angiogenic factors such as vascular Endothelial Growth Factors (VEGFs), Fibroblast Growth Factors (FGFs), Tumor Necrosis Factor-beta (TNF-beta), and Insulin-like Growth Factor-1 (IGF-1). Many of these proteinaceous growth factors are available commercially or can be produced recombinantly using techniques well known in the art.

Alternatively, expression systems comprising vectors, particularly adenovirus vectors, incorporating genes encoding a variety of biomolecules can be dispersed in the invention epoxy-containing PEA polymer compositions and particles thereof for timed release delivery. Methods of preparing such expression systems and vectors are well known in the art. For example, proteinaceous growth factors can be dispersed into the invention bioactive compositions for administration of the growth factors either to a desired body site for local delivery, by selection of particles sized to form a polymer depot, or systemically, by selection of particles of a size that will enter the circulation. Growth factors, such as VEGFs, PDGFs, FGF, NGF, and evolutionary and functionally related biologics, and angiogenic enzymes, such as thrombin, may also be used as bioactive agents in the invention.

Small molecule drugs are yet another category of bioactive agents suitable for dispersion in the invention epoxy-containing PEA polymer compositions and methods of use described herein. Such drugs include, for example, antimicrobials and anti-inflammatory agents as well as certain healing promoters, such as, for example, vitamin A and synthetic inhibitors of lipid peroxidation.

A variety of antibiotics can be dispersed as bioactive agents in the invention epoxy-containing PEA polymer compositions to indirectly promote natural healing processes by preventing or controlling infection. Suitable antibiotics include many classes, such as aminoglycoside antibiotics or quinolones or beta-lactams, such as cefalosporins, e.g., ciprofloxacin, gentamycin, tobramycin, erythromycin, vancomycin, oxacillin, cloxacillin, methicillin, lincomycin, ampicillin, and colistin. Suitable antibiotics have been described in the literature.

Suitable antimicrobials include, for example, Adriamycin PFS/RDF® (Pharmacia and Upjohn), Blenoxane® (Bristol-Myers Squibb Oncology/Immunology), Cerubidine® (Bedford), Cosmegen® (Merck), DaunoXome® (NeXstar), Doxil® (Sequus), Doxorubicin Hydrochloride® (Astra), Idamycin® PFS (Pharmacia and Upjohn), Mithracin® (Bayer), Mitamycin® (Bristol-Myers Squibb Oncology/Immunology), Nipen® (SuperGen), Novantrone® (Immunex) and Rubex® (Bristol-Myers Squibb Oncology/Immunology). In one embodiment, the peptide can be a glycopeptide. “Glycopeptide” refers to oligopeptide (e.g. heptapeptide) antibiotics, characterized by a multi-ring peptide core optionally substituted with saccharide groups, such as vancomycin.

Examples of glycopeptides included in this category of antimicrobials may be found in “Glycopeptides Classification, Occurrence, and Discovery,” by Raymond C. Rao and Louise W. Crandall, (“Bioactive agents and the Pharmaceutical Sciences” Volume 63, edited by Ramakrishnan Nagarajan, published by Marcal Dekker, Inc.). Additional examples of glycopeptides are disclosed in U.S. Pat. Nos. 4,639,433; 4,643,987; 4,497,802; 4,698,327, 5,591,714; 5,840,684; and 5,843,889; in EP 0 802 199; EP 0 801 075; EP 0 667 353; WO 97/28812; WO 97/38702; WO 98/52589; WO 98/52592; and in J. Amer. Chem. Soc. (1996) 118: 13107-13108; J. Amer. Chem. Soc. (1997) 119:12041-12047; and J. Amer. Chem. Soc. (1994) 116:4573-4590. Representative glycopeptides include those identified as A477, A35512, A40926, A41030, A42867, A47934, A80407, A82846, A83850, A84575, AB-65, Actaplanin, Actinoidin, Ardacin, Avoparcin, Azureomycin, Balhimyein, Chloroorientiein, Chloropolysporin, Decaplanin, -demethylvancomycin, Eremomycin, Galacardin, Helvecardin, Izupeptin, Kibdelin, LL-AM374, Mannopeptin, MM45289, MM47756, MM47761, MM49721, MM47766, MM55260, MM55266, MM55270, MM56597, MM56598, OA-7653, Orenticin, Parvodicin, Ristocetin, Ristomycin, Synmonicin, Teicoplanin, UK-68597, UD-69542, UK-72051, Vancomycin, and the like. The term “glycopeptide” or “glycopeptide antibiotic” as used herein is also intended to include the general class of glycopeptides disclosed above on which the sugar moiety is absent, i.e. the aglycone series of glycopeptides. For example, removal of the disaccharide moiety appended to the phenol on vancomycin by mild hydrolysis gives vancomycin aglycone. Also included within the scope of the term “glycopeptide antibiotics” are synthetic derivatives of the general class of glycopeptides disclosed above, including alkylated and acylated derivatives. Additionally, within the scope of this term are glycopeptides that have been further appended with additional saccharide residues, especially aminoglycosides, in a manner similar to vancosamine.

The term “lipidated glycopeptide” refers specifically to those glycopeptide antibiotics that have been synthetically modified to contain a lipid substituent. As used herein, the term “lipid substituent” refers to any substituent contains 5 or more carbon atoms, preferably, 10 to 40 carbon atoms. The lipid substituent may optionally contain from 1 to 6 heteroatoms selected from halo, oxygen, nitrogen, sulfur, and phosphorous. Lipidated glycopeptide antibiotics are well known in the art. See, for example, in U.S. Pat. Nos. 5,840,684, 5,843,889, 5,916,873, 5,919,756, 5,952,310, 5,977,062, 5,977,063, EP 667, 353, WO 98/52589, WO 99/56760, WO 00/04044, WO 00/39156, the disclosures of which are incorporated herein by reference in their entirety.

Anti-inflammatory bioactive agents are also useful for dispersion in used in invention epoxy-containing PEA polymer compositions and methods. Depending on the body site and disease to be treated, such anti-inflammatory bioactive agents include, e.g. analgesics (e.g., NSAIDs and salicyclates), steroids, antirheumatic agents, gastrointestinal agents, gout preparations, hormones (glucocorticoids), nasal preparations, ophthalmic preparations, otic preparations (e.g., antibiotic and steroid combinations), respiratory agents, and skin & mucous membrane agents. See, Physician's Desk Reference, 2005 Edition. Specifically, the anti-inflammatory agent can include dexamethasone, which is chemically designated as (11 [symbol] , 16I)-9-fluro-11,17,21-trihydroxy-16-methylpregna-1,4-diene-3,20-Dione. Alternatively, the anti-inflammatory bioactive agent can be or include sirolimus (rapamycin), which is a triene macrolide antibiotic isolated from Streptomyces hygroscopicus.

The polypeptide bioactive agents included in the invention compositions and methods can also include “peptide mimetics.” Such peptide analogs, referred to herein as “peptide mimetics” or “peptidomimetics,” are commonly used in the pharmaceutical industry with properties analogous to those of the template peptide (Fauchere, J. (1986) Adv. Bioactive agent Res., 15:29; Veber and Freidinger (1985) TINS, p. 392; and Evans et al. (1987) J. Med. Chem., 30:1229) and are usually developed with the aid of computerized molecular modeling. Generally, peptidomimetics are structurally similar to a paradigm polypeptide (i.e., a polypeptide that has a biochemical property or pharmacological activity), but have one or more peptide linkages optionally replaced by a linkage selected from the group consisting of. —CH₂NH—, —CH₂S—, CH₂—CH₂—, —CH═CH— (cis and trans), —COCH₂—, —CH(OH)CH₂—, and —CH₂SO—, by methods known in the art and further described in the following references: Spatola, A. F. in Chemistry and Biochemistry of Amino Acids, Peptides, and Proteins, B. Weinstein, eds., Marcel Dekker, New York, p. 267 (1983); Spatola, A. F., Vega Data (March 1983), Vol. 1, Issue 3, “Peptide Backbone Modifications” (general review); Morley, J. S., Trends. Pharm. Sci., (1980) pp. 463-468 (general review); Hudson, D. et al., Int. J. Pept. Prot. Res., (1979) 14:177-185 (—CH₂NH—, CH₂CH₂—); Spatola, A. F. et al., Life Sci., (1986) 38:1243-1249 (—CH₂—S—); Harm, M. M., J. Chem. Soc. Perkin Trans I (1982) 307-314 (—CH═CH—, cis and trans); Almquist, R. G. et al., J. Med. Chem., (1980) 23:2533 (—COCH₂—); Jennings-Whie, C. et al., Tetrahedron Lett., (1982) 23:2533 (—COCH₂—); Szelke, M. et al., European Appln., EP 45665 (1982) CA: 97:39405 (1982) (—CH(OH)CH₂—); Holladay, M. W. et al., Tetrahedron Lett., (1983) 24:4401-4404 (—C(OH)CH₂—); and Hruby, V. J., Life Sci., (1982) 31:189-199 (—CH₂—S—). Such peptide mimetics may have significant advantages over natural polypeptide embodiments, including, for example: more economical production, greater chemical stability, enhanced pharmacological properties (half-life, absorption, potency, efficacy, etc.), altered specificity (e.g., a broad-spectrum of biological activities), reduced antigenicity, and others.

Additionally, substitution of one or more amino acids within a peptide (e.g., with a D-lysine in place of L-lysine) may be used to generate more stable peptides and peptides resistant to endogenous peptidases. Alternatively, the synthetic polypeptides covalently bound to the biodegradable polymer, can also be prepared from D-amino acids, referred to as inverso peptides. When a peptide is assembled in the opposite direction of the native peptide sequence, it is referred to as a retro peptide. In general, polypeptides prepared from D-amino acids are very stable to enzymatic hydrolysis. Many cases have been reported of preserved biological activities for retro-inverso or partial retro-inverso polypeptides (U.S. Pat. No. 6,261,569 B1 and references therein; B. Fromme et al, Endocrinology (2003)144:3262-3269.

Any suitable and effective amount of the at least one bioactive agent can be released with time from the invention polymer composition, including those in a biodegradable internal fixation device, stent, or dialysis shunt, or in a depot formed from particles thereof introduced in vivo. The suitable and effective amount of the bioactive agent will typically depend, e.g., on the specific alkylene di-acid-containing PEA polymer and type of particle or polymer/bioactive agent linkage, if present. Typically, up to about 100% of the bioactive agent(s) can be released from the invention polymer in vivo. Specifically, up to about 90%, up to 75%, up to 50%, or up to 25% thereof can be released from the polymer. Factors that typically affect the release rate from the polymer are the types of polymer/bioactive agent linkage, and the nature and amount of additional substances present in the formulation, as well as the chemical structure of the polymer itself.

In addition to humans, the invention epoxy-containing PEA polymer compositions, as well as particles and medical devices fabricated therefrom, are also intended for use in veterinary practice, including a variety of mammalian patients, such as pets (for example, cats, dogs, rabbits, and ferrets), farm animals (for example, swine, horses, mules, dairy and meat cattle) and race horses.

The compositions used in the invention devices and methods of delivery may comprise an “effective amount” of one or more backbone bioactive agent(s) and optional bioactive agents of interest. That is, an amount of a bioactive agent will be incorporated into the polymer that will produce a sufficient therapeutic or palliative response in order to prevent, reduce or eliminate symptoms. The exact amount necessary will vary, depending on the subject to which the composition is being administered; the age and general condition of the subject; the capacity of the subject's immune system, the degree of therapeutic or palliative response desired; the severity of the condition being treated or investigated; the particular bioactive agent selected and mode of administration of the composition, among other factors. An appropriate effective amount can be readily determined by one of skill in the art. Thus, an “effective amount” will fall in a relatively broad range that can be determined through routine trials. For example, for purposes of the present invention, an effective amount will typically range from about 1 μg to about 100 mg, for example from about 5 μg to about 1 mg, or about 10 μg to about 500 μg of the bioactive agent delivered.

The following examples are meant to illustrate, but not to limit, the invention.

### EXAMPLE 1

Synthesis of di-p-nitrophenyl-trans-epoxysuccinate Monomer

Trans-epoxysuccinic acid was synthesized by the treatment of fumaric acid with hydrogen peroxide in the presence of sodium tungstate as catalyst, as previously described (Payne G. B., Williams P. H. J. Org. Chem. (1959) 24:54-55). The acid was collected in 60% yield (scheme 2.A). Elemental analysis: [C₄H₄O₅] calcd. C: 36.38%, H: 3.05%; found C: 36.63%, H: 3.45%. ¹H NMR (DMSO-d₆/CDCl₃ (1/3 v/v), 300 MHz, δppm): 11.55 (s, broad, 2H, —COOH), 3.41 (s, 2H).

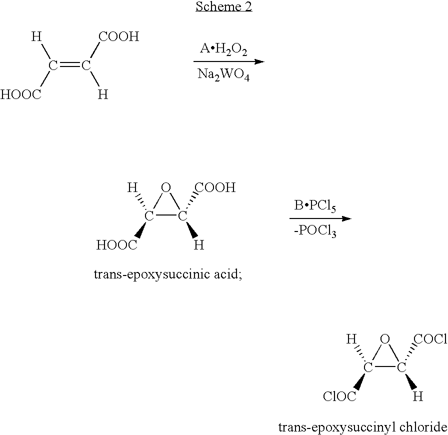

Formed epoxy-di-acid was transformed into the corresponding dichloride, using PCl₅ (scheme 2.B). After removing the by-product (POCl₃) under reduced pressure, the dichloride crystallized at room temperature. Recrystallization from light petroleum yielded the dichloride in ca. 80% yield (per epoxy-di-acid); m.p. 51-53° C. lit. m.p. 50-52° C. (Campbell T. W and McDonald R. N. J. Polymer. Sci., A (1963) 1:2525-2535). The FTIR spectrum (Nujol) showed strong carbonyl absorption at 1766 cm⁻¹ (vs. 1709 cm⁻¹ in the corresponding di-acid), which showing confirms the presence of COCl groups.

The goal monomer, di-p-nitrophenyl-trans-epoxysuccinate (compound V.4), was obtained using the Shotten-Bauman procedure—by interfacial reaction of a chloroform solution of dichloride with p-nitrophenol and Na₂CO₃ in water (scheme 3). 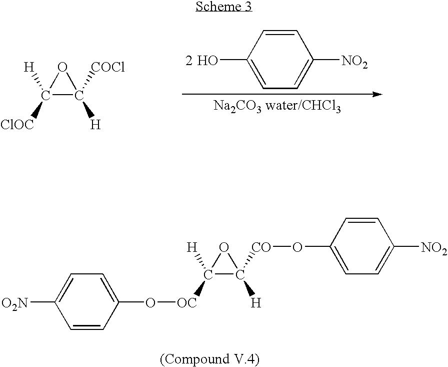
The product (compound V.4) was obtained in ca. 90% yield per starting dichloride, with m.p. 182-184° C. (from light petroleum). Elemental analysis confirmed the assumed structure: [C₁₆H₁₀N₂O₉] Calcd. C: 51.35%, H: 2.69%, N: 7.49%; found C: 51.01%, H: 2.43%, N: 7.30%. ¹H NMR (DMSO-d₆/CDCl₃ (1/3 v/v), 300 MHz, δ ppm): 8.33 (d, 4H, J=9.1 Hz), 7.54 (d, 4H), 4.23 (s, 2H). The FTIR (Nujol) spectrum showed a strong C═O absorption at 1758 cm⁻¹, indicating the presence of the p-nitrophenyl ester group.

Other known techniques for forming esters, e.g. the N,N′-carbonyldiimidazole method, were also implemented for synthesis of compound V.4 as indicated in scheme 4: 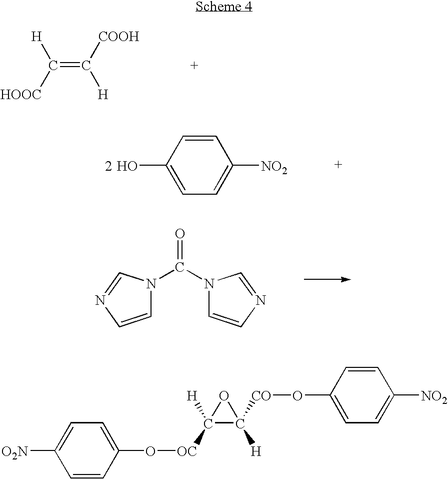
The yield of the diester (compound V.4) by this scheme was ca. 82%, which is somewhat higher than yield of the diester yield via dichloride (ca. 72% per di-acid).
Synthesis of di-p-nitrophenyl-cis-epoxysuccinate Monomer

Di-p-nitrophenyl-cis-epoxysuccinate (Compound V.5) was synthesized in a manner analogous to that described above for the trans-isomer. First, cis-epoxysuccinic acid was prepared from maleic acid, by the treatment with hydrogen peroxide and sodium tungstate, as described above Payne G. B. and Williams P. H., supra). El. analysis: C₄H₄O₅, calcd. C: 36.38%, H: 3.05%; found C: 36.57%, H: 3.36%.

Obtained cis-epoxy succinic acid (scheme 5) was transformed into the corresponding dichloride using PCl₅ following Campbell and McDonald's procedure, supra:

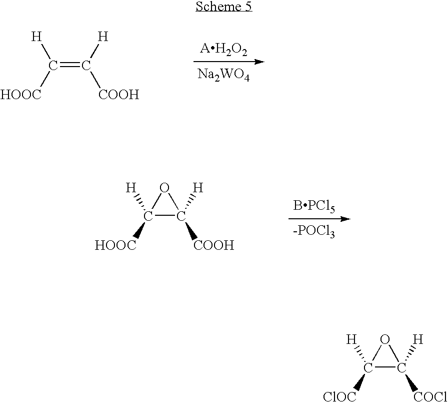

The goal di-p-nitrophenyl-cis-epoxy succinate (Compound V.5) was synthesized by interfacial interaction of a vigorously stirred solution of dichloride in carbon tetrachloride with an aqueous solution of p-nitrophenol and sodium carbonate:

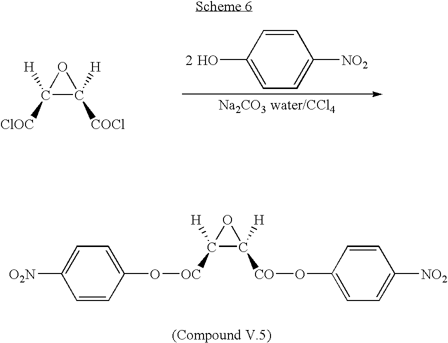

The goal compound V.5 was obtained in a good yield (ca. 90% per dichloride). After recrystallization from acetone, the yield was decreased to 62%, and m.p. was 184-186° C., which result is very close to the m.p. of trans-isomer (182-184° C.). The mixed sample of these two isomers however, melted at 155-160° C., indicating that the two are different compounds. ¹H NMR (DMSO-d₆/CDCl₃ (1/3 v/v), 300 MHz, δ ppm): 8.30 (d, 4H, J=9.1 Hz), 7.41 (d, 4H), 4.33 (s, 2H). El analysis: [C₁₆H₁₀N₂O₉], calcd. C: 51.35%, H: 2.69%, N: 7.49%; found C: 51.16%, H: 2.49%, N: 7.38%.

### EXAMPLE 2

Solution Active Polycondensation (APC)

Epoxy PEA synthesis on the basis of di-p-nitrophenyl-trans-epoxy succinate (Table 1.1) Polycondensation of the new active diester V.4 with di-p-toluenesulfonic acid salts, for example, bis-(L-phenylalanine)-1,6-hexylene diester and/or bis-(L-leucine)-1,6-hexylene diester was carried out in N,N-dimethylacetamide (DMA) in the presence of triethylamine as a p-toluenesulfonic acid acceptor. The reaction with V.4 was carried out at room temperature. Significant exothermal effect was observed, which was assigned to increased reactivity of diester V.4, since it is known that hetero-atoms in α-position sharply increase (e.g. by about 3 orders of magnitude in the case of methyl esters) the reactivity of esters of dicarboxylic acids (R. D. Katsarava. (1991) Uspekhi Khimii (Russian Chem. Rev), 60:1419).

Synthesis on the basis of di-p-nitrophenyl-cis-epoxy succinate (Table 1.1) Epoxy-PEA synthesis based on a cis-isomer was carried out similar to above trans-isomer: The compound V.5 was reacted with di-p-toluenesulfonic acid salt of bis-(L-phenylalanine)-1,6-hexylene diester and/or bis-(L-leucine)-1,6-hexylene di-ester at room temperature in DMA, in the presence of triethylamine as acid acceptor.

New co-PEAs based on multiple di-carboxylic acids were also synthesized under the same solution polycondensation conditions. The monomer ratio in reaction mixture, for example di-p-nitrophenyl-cis-epoxy succinate (V.5) (40 mol %) and di-p-nitrophenyl sebacate (60 mol %), agreed with the final co-PEA composition as determined by ¹H NMR.

It should be noted that the reaction solutions in all cases were rather viscous. However, after precipitation in water the solution viscosity of purified cis-epoxy polymers was as shown in Table 1.1.

Partial gelation was observed during extended condensation time for trans-epoxy PEAs; whereas no gel was-formed in reaction solutions of cis-isomers. Gel-formation can be ascribed to interaction of terminal amino groups of the growing chains with epoxy-groups of the polymer backbones, leading to 3D networks. Epoxy-PEAs with un-protected terminal amino groups were found to undergo auto-crosslinking and lose solubility about two weeks after separation from the reaction solutions. In order to avoid crosslinking, the terminal amino groups of macromolecules were protected by adding p-nitrophenyl acetate or acetic anhydride at the final stage.

Interfacial Polycondensation

The cis-isomers of the invention epoxy-PEAs were also synthesized by a method known as “Interfacial Polycondensation,” proceeding at the interface of a hydrophobic organic solvent and water. This method applies di-acid dichlorides instead of active diesters. The reaction in this method is fast and is completed within 5-15 min. Obtained polymers were summarized in Table 1.2.

It was found that the interfacial method was unsuitable for synthesizing high molecular weight PEAs based on cis-epoxy succinic acid. However, a mixture of cis-epoxy succinyl dichloride (40 mol. %) and the other hydrophobic diacid chloride, sebacoylchloride (60 mol. %), yielded a relatively high molecular weight copolymers (η_{red} up to 0.48 dL/g; DMF, c=0.5 g/dL) with good film-forming properties. Here also, polymers with un-protected terminal amino groups lost solubility (swelled) in organic solvents about two weeks after isolation. The polymers were more stable in chloroform solutions and no cross-linkage was observed after more than four weeks.

TABLE 1.2
Epoxy-poly(ester amide)s obtained by interfacial polycondensation¹⁾.

| | | Yield, | η_{red}²⁾, | Solubility³⁾ | | |
|---|---|---|---|---|---|---|
| # | Polymer | in % | in dL/g | Acetone | Chloroform | DMF |
| 1 | [8-L-Phe-6]_{0.6}- | 78.5 | 0.48 | ± | + | + |
| | [t-ES-L-Phe-6]_{0.4} | | | | | |
| 2 | t-ES-L-Leu-6 | 56.8 | 0.10 | ± | + | + |
| 3 | [8-L-Leu-6]_{0.5}- | 82.1 | 0.21 | − | + | + |
| | [t-ES-L-Leu-6]_{0.5} | | | | | |
| 4 | [8-L-Leu-6]_{0.8}- | 81.1 | 0.23 | − | + | + |
| | [t-ES-L-Leu-6]_{0.2} | | | | | |
| 5. | [8-L-Phe-6]_{0.6}- | 81.5 | 0.22 | ± | + | + |
| | [c-ES-L-Phe-6]_{0.4} | | | | | |
| 6. | c-ES-L-Leu-6 | 87.5 | 0.09 | ± | + | + |
| 7. | [8-L-Leu-6]_{0.8}- | 83.0 | 0.12 | ± | + | + |
| | [c-ES-L-Leu-6]_{0.2} | | | | | |

¹⁾Quantitative determinations of the monomer ratio in final co-PEA compositions were not conducted.
²⁾Determined in DMF at c = 0.5 g/dL and t = 25° C.
³⁾<<+>> - soluble, <<±>> - partially soluble.

### EXAMPLE 3

Transformations of Epoxy PEAs

Interaction with Amines: a Model Study. Interaction with Primary Amines.

To study the interaction of epoxy-PEAs with primary amines, monoamine-N-(6-aminohexyl)-2,4-dinitroanilin (Compound 6), containing a “reporting group” was prepared; reporting group 2,4-dinitroanilin chromophore group absorbs strongly at about 360-400 nm and can be readily monitored by photometry. 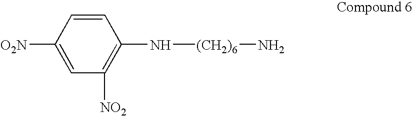
Compound 6 was synthesized by interacting hexamethylene diamine (1.0 mole) with 2,4-dinitrofluoro benzene (1.0 mole) in DMF in the presence of triethylamine as an acceptor of HF.

Epoxy-PEA, t-ES-L-Leu-6 (I.1) was selected for this study because of the absence of absorbance in UV and VIS regions of the spectrum. The assumed scheme of the reaction is shown below, in Scheme 7:

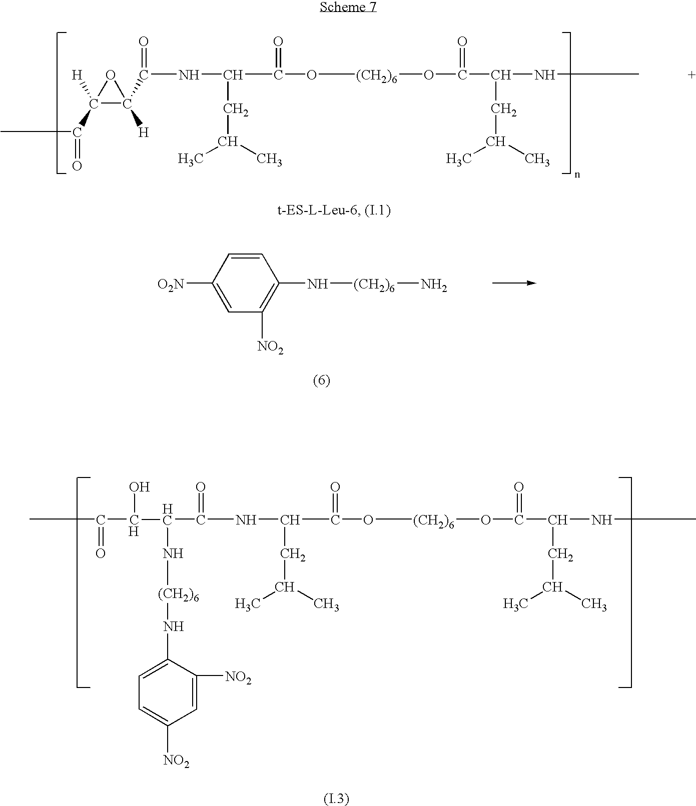

The polymer solution in a DMF/DMSO mixture was heated to 100° C. The mole ratio of epoxy-groups to primary amino groups was 1:2. Under these conditions the reaction proceeded homogeneously; however, after cooling to room temperature, excess of compound 6 was crystallized out. The supernatant was dialyzed against DMF until the outer solution became colorless while inside the dialysis bag the solution retained a brownish-yellow color, a result that indirectly speaks for formation of the intended polymer adduct (scheme 7). The dialyzed product was precipitated in water and dried. FTIR-data of the conjugated poly(ester amide) showed strong absorption bands at 1740 cm¹ (ester CO) and 1660-1665 cm⁻¹ (amide CO) (cf. with FTIR spectrum of the starting epoxy-poly(ester amide) t-ES-L-Leu-6). Coincidence of the UV-spectrum of this compound with the UV-spectrum of amine 6 (2,4-dinitroanilin chromophore) speaks for the assumed structure of PEA-conjugate (1.3).

Covalent Attachment of Bioactive Agent to Epoxy-Containing PEA

The covalent attachment of 4-amino-TEMPO to the PEA-t-ES-L-Leu-6 polymer was conducted. This polymer was selected because it does not absorb in UV-region 240-280 nm, whereas a 4-amino-TEMPO free radical absorbs UV rather strongly at 267 nm.

For covalent attachment, 4-amino-TEMPO and t-ES-L-Leu-6 (I.1) were dissolved in mole ratio 1.2:1.0 in DMF (10% solution by polymer) and the solution was stirred at 60° C. for 36 h. The assumed scheme of the reaction is shown below (Scheme 8):

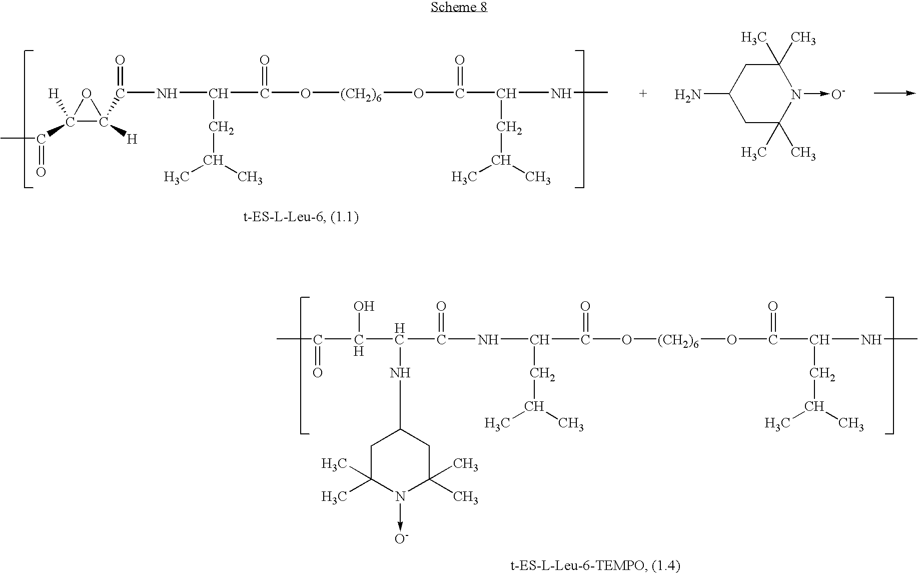

The reaction solution was poured into water; the precipitated polymer was thoroughly washed with 50% ethanol, dissolved in ethanol (95%) and dialyzed against ethanol until the absorption at 267 nm of the outer phase disappeared. The content of the dialysis bag was poured into water, the precipitated polymer was dried. A UV-spectrum of adduct t-ES-L-Leu-6-TEMPO (I.4) was recorded in DMF solution. A strong absorption at 267 nm, corresponding to iminoxyl radical, confirmed formation of polymer-4-amino-TEMPO conjugate (I.4).

Other evidence for formation of the adduct was obtained by gel-chromatography (eluent 0.1 N LiBr in DMF, c=1×10⁻³ mole/L) using both refractive index (RI) and ultraviolet (UV) detectors. Studies showed that the adduct formation started at room temperature after t-ES-L-Leu-6 and 4-amino-TEMPO were mixed in DMF solution. Conjugation was completed at 60° C. after 36 h of interaction.

### EXAMPLE 4

Modification of Epoxy-PEA with (meth)acryloyl Groups

Approach 1: PEAs with crosslinkable (meth)acryloyl pendent groups were synthesized using different approaches. In the first approach the functional photosensitive polymers were developed by a two-step method (scheme 9). In the first step, PEA t-ES-L-Leu-6 was interacted with secondary amine, di-butyl amine, resulting in poly(ester amide) with lateral hydroxyl and tert-amino groups. In the second step, this molecule was acylated using acryloyl chloride.

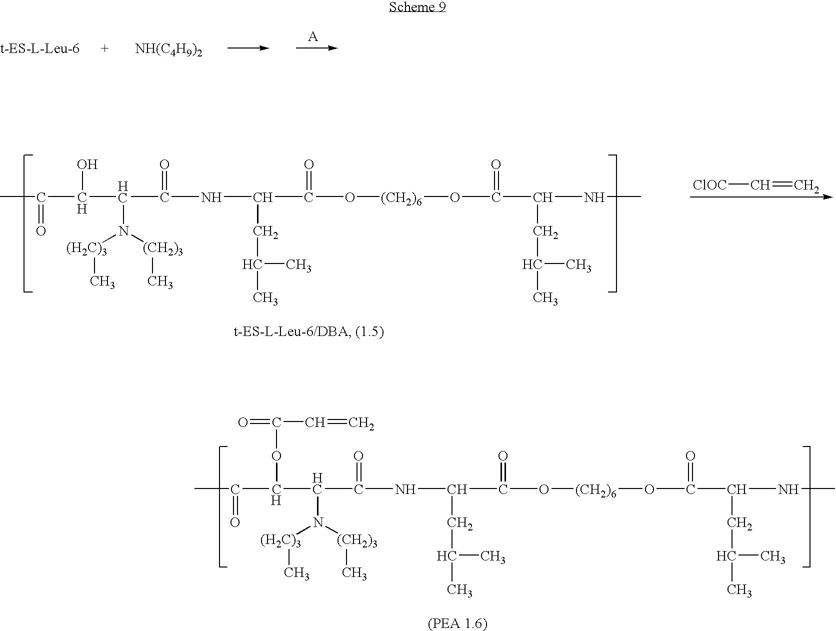

In a typical procedure, a solution of the polymer (I.1) in DMF (20% solution w/v) was treated at 50° C. for 24 hours by di-butyl amine (mole ratio epoxy groups: di-butyl amine=1:2, Scheme 9). The obtained polymer was precipitated in water, filtered off, dried, redissolved in DMF, and treated at 0-5° C. with acryloyl chloride (mole ratio elemental unit of the polymer:acryloyl chloride was 1:1.2). The final polymer was precipitated and washed in water, and dried. The formation of acrylic ester was confirmed by UV-spectroscopy: both the starting polymer t-ES-L-Leu-6 (I.1) and its adduct t-ES-L-Leu-6/DBA (I.5) did not absorb in the UV region; whereas, after the treatment with acryloyl chloride, an absorption maximum at 265 nm was detected, a result specific for acryloyl groups.

Approach 2: A one-step synthesis of PEAs with unsaturated lateral groups was carried out by direct interaction of PEA (I.1) with acryloyl chloride. The intended adduct was formed after the treatment of t-ES-L-Leu-6 with acryloyl chloride (mole ratio oxirane/acryloyl chloride 1:2) in DMA solution at room temperature for 12 h, with subsequent precipitation and washing of the polymer with water until a negative reaction with Cl⁻ ions was observed. The reaction scheme (Scheme 10) is as follows:

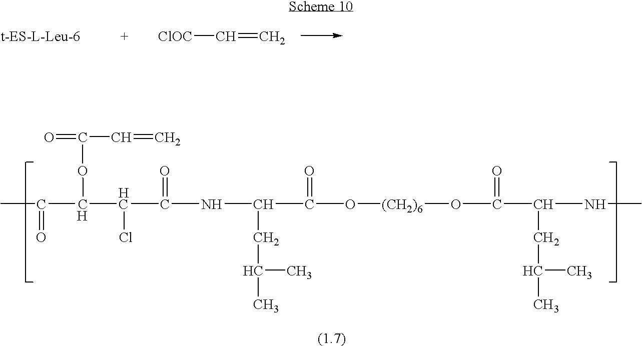

An absorption maximum of acrylic acid conjugate at 265 nm was observed in UV spectra of the reaction product PEA-acrylate I.7.

At the same time this product gave a positive reaction on halogen, possibly indicating existence of covalently bound chlorine in the polymer molecules. Elemental analysis showed that about ⅓ of the epoxy groups of the polymeric backbones had interacted with acryloyl chloride (calculated using data of analysis of element Cl). El. Analysis calculated for 33% epoxy-group conversion: C: 58.84%, H: 7.91%, N: 5.99%, Cl: 2.50%; found C: 58.29%, H: 7.82%, N: 5.74%, Cl: 2.14%.

Possible mechanisms of the reaction. A nucleophilic mechanism of the reaction consists in the direct interaction of the oxygen atom of the oxirane cycle with the carbonyl group of the acid chloride, followed by the formation of the goal adduct, as is shown in the reaction scheme below: 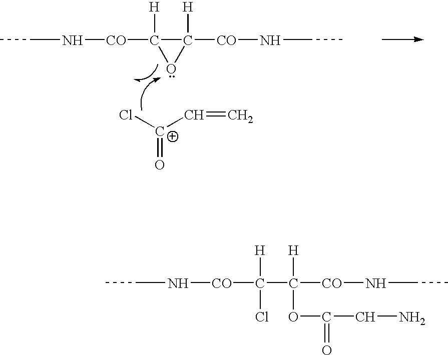
According to an electrophilic mechanism of the reaction, a complex ([C⁺] . . . Cl⁻) of acryloyl chloride with solvent (DMA) is formed at the first stage of the reaction, leading to the generation of Cl⁻ ions: 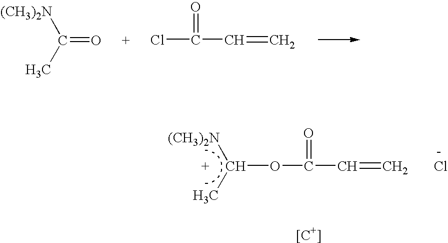
Then, Cl⁻ ion attacks the oxirane cycle and opens it; the alkoxide ion formed is rapidly acylated with cation [C⁺] and leads to the formation of the goal adduct:

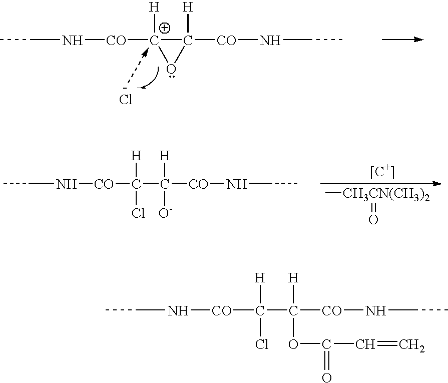

Approach 3. In this approach, it was assumed that the electrophilic mechanism is more efficient. Accordingly, the interaction of t-ES-L-Leu-6 with sodium salt of methacrylic acid was carried out in DMF at 60° C. for 24 h. (Scheme 11):

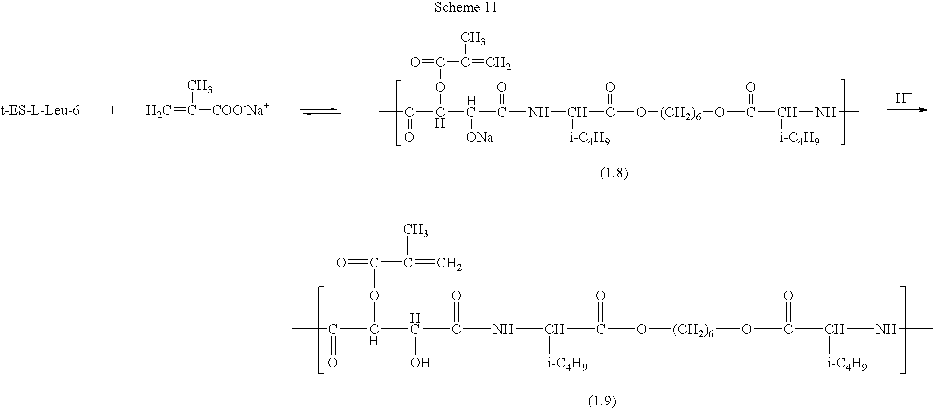

The resulting reaction solution was poured into water and the separated polymer was washed thoroughly again with water to remove residual methacrylic acid and its salt. Thereafter the polymer was dried and re-precipitated from DMF into water and dried again. The presence of double-bond absorption signal in the UV spectrum of the test sample and the absence in the spectrum of the control polymer (I.1) confirmed the covalent conjugation.

All the polymers obtained in Example 4, regardless of the scheme of synthesis, underwent cross-linkage in the presence of radical initiators, a result that also suggests the presence in the molecules of (meth)acryloyl reactive groups.

Another modification of epoxy PEA. The synthesis of active derivatives of the invention epoxy-PEAs has also been carried out. For this purpose, a PEA with hydroxyl groups—t-ES-L-Leu-6/DBA, (I.5, Scheme 9), was treated with p-nitrophenylchloroformate (p-NPC) in DMA solution to yield Compound I.10, scheme 12:

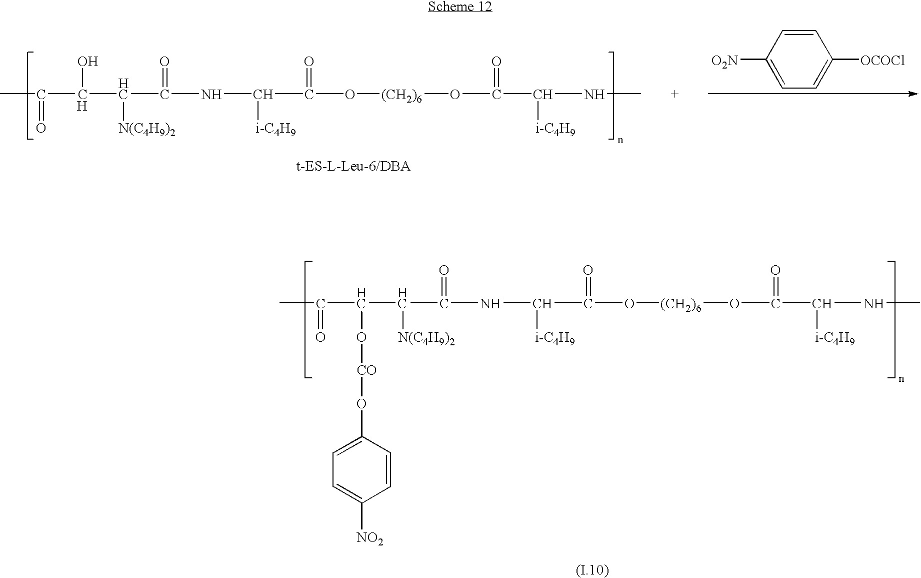

The resulting polymer I.10, which contains lateral active carbonate groups, was separated from the reaction solution by precipitation into water acidified to pH 3-4, polymer was filtered off, thoroughly washed with water and dried at room temperature under reduced pressure.

Formation of the polymer with active carbonate groups was confirmed by UV-spectrophotometer by showing that activated polymer I.10 absorbs in the UV region of the spectrum while the starting polyol, t-ES-L-Leu-6/DBA, does not.

Activated polymer I.10 was interacted with free iminoxyl radical, 4-amino-TEMPO, which bound to the polymer via a urethane link as shown in Scheme 13 below:

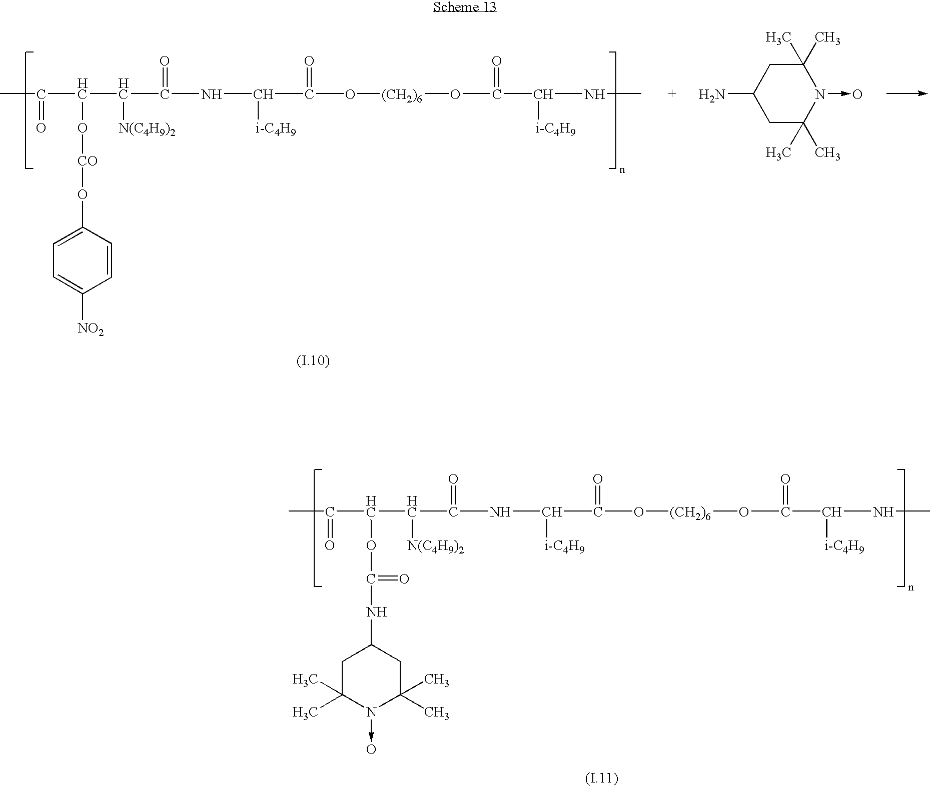

For this reaction, 1 g of obtained active polymer (I.10) was dissolved in 10 mL of DMF at room temperature and then 0.5 g of 4-amino-TEMPO was added. The homogeneous solution was stirred for 1 h and kept at room temperature overnight. The resulting polymer was precipitated in distilled water, washed with water, and dried at room temperature under reduced pressure. The covalent attachment of the iminoxyl radical was confirmed by GPC using two detectors (RI and UV).

Curing of Epoxy-Containing PEAs

Thermal curing:_It was found that epoxy-PEAs undergo cross-linkage after heating in the range 100-150° C., as confirmed by the loss of solubility and formation of gel in organic solvents. It was found that epoxy-PEAs undergo cross-linkage after heating in the range 100-150° C., as confirmed by the loss of solubility and formation of gel in organic solvents.

It was found that epoxy-PEAs undergo cross-linkage after heating in the range 100-150° C., as confirmed by the loss of solubility and formation of gel in organic solvents.

Thermal curing of epoxy-PEA was studied using IR-spectroscopy. The reactions were monitored by the change of intensity of the absorption band at 890 cm⁻¹ ascribed to oxirane cycle (L. J. Bellamy, The Infra-Red Spectra of Complex Molecules, (Russian translation), Izd. Inostrannoi Literatury, Moscow, 1963, p. 590). Absorption bands at 1460 and 1530 cm⁻¹ were used as internal standards to estimate the curing degree.

Obtained data showed that about 70% of the epoxy groups were transformed upon heating at 120° C. for 100 h. The solubility tests showed that crosslinking was occurred at a relatively early stage (after 6 h).

Photochemical curing. The invention epoxy-PEA films underwent photoi.e., curing after UV-exposure, as was confirmed by loss of solubility in organic solvents such as chloroform, ethanol, and DMF.

The photochemical reactions of invention epoxy-PEAs, initiated by intense broad-band UV light, underwent cross-linking without use of specific catalysts (cationic photoinitiators) normally used for photochemical transformations of epoxy-monomers and oligomers (S. K. Rajaraman et al. (1999). J. Polym. Sci. Part A. Polym. Chem. 37, 4007 and Z. Gomurashvili et al. (2001) J. Polym. Sci. Part A: Polym. Chem. 39, 1187-1197).

The non-catalytic photochemical transformation of epoxy-containing PEAs is important in medicinal applications because there is no need to use toxic catalysts that are difficult to be removed from cured polymers.

Chemical curing in the presence of aliphatic diamines 0.1 g of epoxy-polymer (I.1) and 1,6-hexamethylene diamine (as fatty diamine, 10% w/w) was dissolved in 2 mL of chloroform, cast onto a smooth hydrophobic surface and left overnight under atmospheric conditions while chloroform was evaporated up to dryness. An elastic film insoluble in chloroform (swelled only) was obtained.

### EXAMPLE 5

Synthesis of Biodegradable Three-Dimensional Hybrid Hydrogel Networks

For synthesis of biodegradable three-dimensional hybrid hydrogel networks, it was necessary to have reactive derivatives on both PEAs and polysaccharides.

Methacryloyl dextran (MaDX) was chosen as an active partner of PEA (I.6). MaDX was prepared as described by Chu et al. J. Biomed Mater Res (2000) 49:517) by interaction of dextran (DX) (from Leuconostoc mesenteroides, Sigma Chemicals) with methacrylic acid anhydride (Lancaster Chemicals) in DMF/LiCl solution with 1:1 proportion of OH-groups of dextran to methacrylic acid anhydride.

For preparation of hybrid hydrogels, the MaDX and polymethacryloyl-PEA I.6, in a weight ratio of 90:10 were dissolved in DMF, the solution was cast onto Petri dishes, and the solvent was evaporated up to dryness under vacuum. Two experiments were performed, one without initiator and a second, using 1% w/w benzoyl peroxide per mixture of MaDX and PEA I.6. The films obtained after the removal of DMF were heated at 80° C. for 8 h. In both cases films became insoluble and only swelled in water, indicating the formation of three-dimensional networks. When mixture of the polymer was not subjected to thermal treatment and placed in water, complete dissolution of dextran and precipitation of the PEA was observed.

Other weight ratio mixtures of Dextran to PEA I.6 were also prepared: 95:5, 90:10, 75:25 and 50:50. The mixtures were dissolved in DMF (1 g in 10 mL) in the presence of 1% w/w benzoyl peroxide, cast onto Petri dishes, and the solvent was evaporated up to dryness. After the removal of DMF, obtained films were heated at 100° C. for 8 h. In all cases, polymers became insoluble and only swelling in water was observed, indicating formation of three-dimensional networks.

FIG. 1 illustrates swelling degrees of gels, estimated by water uptake in weight %. As can be seen from these data, water uptake is rather high, up to 50% content of PEA I.6. However, transparent gels were obtained only at weight ratios 95:5 and 90:10 of MaDX:PEA I.6.

Hydrogels were also obtained by direct interaction of epoxy-containing PEAs with dextran (un-modified) in the presence of both metallic sodium and sodium methylate. In preliminary experiments, hydrogels with a high swelling index (up to 300-900%) but with low yields (6-20%) were obtained.

### EXAMPLE 6

In Vitro Biodegradation of Epoxy-PEAs

In vitro biodegradation of invention epoxy-PEAs was carried out in the presence of hydrolases. α-Chymotrypsin (Fluka, Basel, Switzerland), with activity 300-340 ATEE/mg enzyme (ATEE—(Acetyl Tyrosine Ethyl Ester, Sigma Chemicals) and lipase (Wako Pure Chemicals, Wako, Tex.), with activity 50-60 triacetin (units/mg enzyme) were used in these experiments. Polymeric circular disks with diameter of 4 cm and weight 500-600 mg were placed at 37° C. in 10 mL of 0.2 M phosphate buffer with pH 7.4 containing 4 mg of either of these enzymes. Biodegradation was monitored by weight loss in mg/cm², by removing the disk from the buffer solution, thoroughly washing with water and drying at 50° C. up to constant weight. This procedure was repeated every 24 h (5 times, total incubation time 120 h) using, in each case, a freshly prepared enzyme solution.

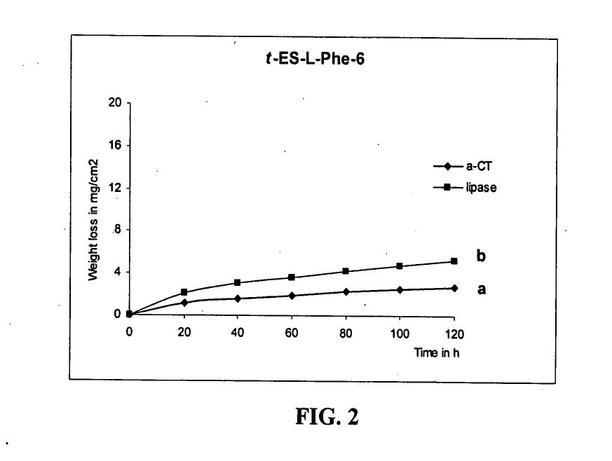

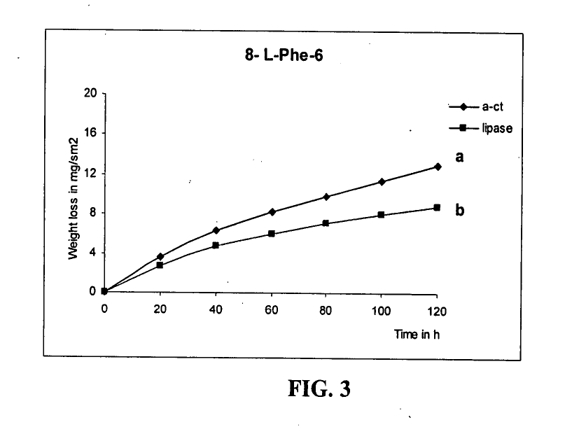

The experimental data are summarized in FIG. 2. From these data it can be concluded that epoxy-containing PEA t-ES-L-Phe-6 (I.1) undergoes enzyme-catalyzed biodegradation at a rate of about 30% (with a-chymotrypsin) and about 50% (with lipase) of the biodegradation rate of aliphatic poly(ester amide) 8-L-Phe-6 (FIG. 3). The PEA 8-L-Phe-6 of formula 1, with R¹═(CH₂)₈, R³═CH₂C₆H₅, and R⁴═(CH₂)₆ exhibited the most rapid biodegradation rates among the same family of PEAs tested (G. Tsitlanadze, supra and G. Tsitlanadze et al. J. Mater Sci.: Mater in Medicine (2004) 15:185-190). It has to also be noted that epoxy-containing PEA t-ES-L-Phe-6 (I.1) undergoes lipase-catalyzed biodegradation faster than (x-chymotrypsin-catalyzed biodegradation.

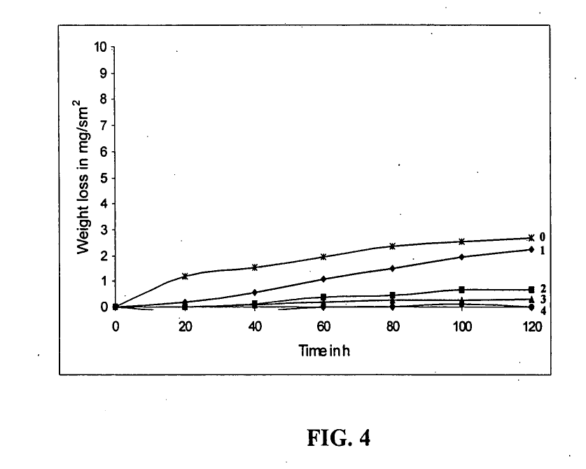

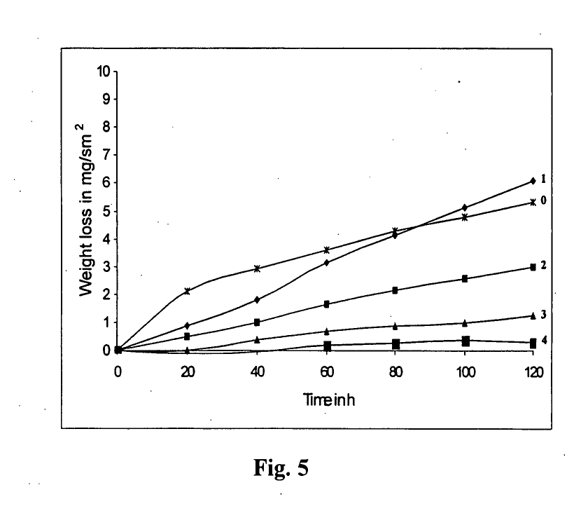

In vitro biodegradation of epoxy-PEA films prior and after various degrees of thermal curing were also conducted. The results summarized in FIGS. 4 and 5 show that the rates of enzyme-catalyzed hydrolysis for both α-chymotrypsin and lipase decrease substantially with an increased degree of cross-linking. This reduction of biodegradation rates could be ascribed to the decreased mobility of macromolecules after crosslinking.

All publications, patents, and patent documents are incorporated by reference herein, as though individually incorporated by reference. The invention has been described with reference to various specific and preferred embodiments and techniques. However, it should be understood that many variations and modifications might be made while remaining within the spirit and scope of the invention.

Although the invention has been described with reference to the above examples, it will be understood that modifications and variations are encompassed within the spirit and scope of the invention. Accordingly, the invention is limited only by the following claims.

What is claimed is:

1. A biodegradable polymer composition comprising a poly(ester amide) (PEA) polymer having a chemical formula described by general structural formula (I), 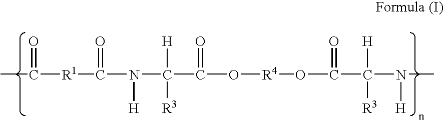
wherein n ranges from about 15 to about 150; R¹ in at lest one individual n unit is epoxy-(C₂-C₁₂)alkylene, while additional R¹s are independently selected from (C₂-C₂₀)alkylene, (C₂-C₂₀)alkenylene, α,ω-bis(4-carboxyphenoxy)-(C₁-C₈)alkane, 3,3′-(alkanedioyldioxy)dicinnamic acid or 4,4′-(alkanedioyldioxy)dicinnamic acid, α,ω-alkylene dicarboxylates of formula (III) below or saturated or unsaturated residues of therapeutic di-acids; whereas R⁵ and R⁶ in Formula (III) are independently selected from (C₂-C₁₂)alkylene or (C₂-C₁₂)alkenylene; the R³s in individual n units are independently selected from the group consisting of hydrogen, (C₁-C₆)alkyl, (C₂-C₆)alkenyl, (C₂-C₆)alkynyl, (C₆-C₁₀)aryl (C₁-C₆)alkyl, and —(CH₂)₂SCH₃; and R⁴ is independently selected from the group consisting of (C₂-C₂₀)alkylene, (C₂-C₂₀)alkenylene, (C₂-C₈)alkyloxy, (C₂-C₂₀)alkylene, bicyclic-fragments of 1,4:3,6-dianhydrohexitols of structural formula (II), saturated or unsaturated therapeutic diol residues, and combinations thereof; 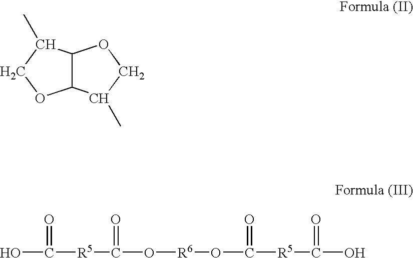
or a PEA polymer having a chemical formula described by structural formula (IV) 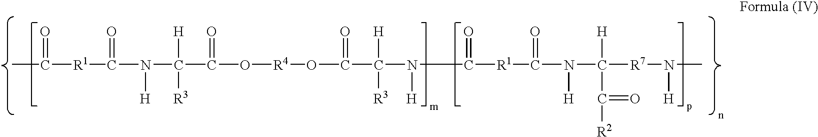
wherein n ranges from about 15 to about 150, m ranges about 0.1 to 0.9; p ranges from about 0.9 to 0.1; R¹ in at least one individual n or m unit is epoxy-(C₂-C₁₂)alkylene while additional R¹s are independently selected from (C₂-C₂₀)alkylene and (C₂-C₂₀)alkenylene, α,ω-bis(4-carboxyphenoxy)-(C₁-C₈)alkane, 3,3′-(alkanedioyldioxy)dicinnamic acid, 4′-(alkanedioyldioxy)dicinnamic acid, or α,ω-alkylene dicarboxylates of structural formula (III) or saturated or unsaturated residues of therapeutic di-acids;
whereas R⁵ and R⁶ in Formula (III) are independently selected from (C₂-C₁₂)alkylene or (C₂-C₁₂)alkenylene; each R² is independently hydrogen, (C₁-C₁₂)alkyl, (C₆-C₁₀)aryl or a protecting group; the R³s in individual m monomers are independently selected from the group consisting of hydrogen, (C₁-C₆)alkyl, (C₂-C₆)alkenyl, (C₂-C₆)alkynyl, (C₆-C₁₀)aryl (C₁-C₆)alkyl, and —(CH₂)₂SCH₃; R⁴ is independently selected from the group consisting of (C₂-C₂₀)alkylene, (C₂-C₂₀)alkenylene, (C₂-C₈)alkyloxy, (C₂-C₂₀)alkylene, bicyclic-fragments of 1,4:3,6-dianhydrohexitols of structural formula (II), residues of saturated or unsaturated therapeutic diols and combinations thereof; and R⁷ is independently (C₂-C₂₀)alkyl or (C₂-C₂₀)alkenyl.

2. The composition of claim 1, wherein the at least one R¹ is (C₂-C₁₂)epoxy-alkylene.

3. The composition of claim 1, wherein the PEA is a homo-polymer.

4. The composition of claim 1, wherein the PEA is a co-polymer.

5. The composition of claim 1, wherein the composition further comprises a non-epoxy-containing biocompatible, polymer.

6. The composition of claim 5, wherein the non-epoxy-containing biocompatible polymer is a PEA.

7. The composition of claim 5, wherein the non-epoxy-containing biocompatible polymer is a poly(ester urea) or a poly(ester urethane).

8. The composition of claim 1, wherein R¹s in at least one of the n or m units is independently selected from residues of α,ω-alkylene dicarboxylates of formula (III) wherein R⁵ and R⁶ in Formula (III) are independently selected from (C₂-C₁₂)alkylene or (C₂-C₁₂)alkenylene or the R¹s in at least one of the n or m units is selected from (C₂-C₂₀)alkylene and (C₂-C₂₀)alkenylene, α,ω-bis(4-carboxyphenoxy)-(C₁-C₈)alkane, 3,3′-(alkanedioyldioxy)dicinnamic acid, 4′-(alkanedioyldioxy)dicinnamic acid, or saturated or unsaturated residues of therapeutic di-acids.

9. The composition of claim 1, wherein at least one R¹ is a saturated or unsaturated residue of a therapeutic diacid.

10. The composition of claim 1, wherein the R³ s in an n or m unit are independently selected from hydrogen, CH₂—CH(CH₃)₂, CH₃, CH(CH₃)₂, CH(CH₃)—CH₂—CH₃, CH₂—C₆H₅, or (CH₂)₂SCH₃.

11. The composition of claim 1, wherein all of the R³s are selected from hydrogen, CH₂—CH(CH₃)₂, CH₃, CH(CH₃)₂, CH(CH₃)—CH₂—CH₃, CH₂—C₆H₅, —(CH₂)₃, or (CH₂)₂SCH₃.

12. The composition of claim 1, wherein the composition biodegrades in a bioenzyme over a period of about two weeks days to about six months.

13. The composition of claim 1, wherein the PEA is unsaturated and the polymer undergoes cross-linking upon exposure to heat in the range from about 100° C. to about 150° C.

14. The composition of claim 1, wherein the PEA is unsaturated and the polymer undergoes photochemical cross-linking without use of a chemical catalyst.

15. The composition of claim 14, wherein the photochemical cross-linking is by exposure to UV light.

16. The composition of claim 15, wherein the cross-linked composition is fabricated in the form of a biodegradable implantable medical device.

17. The composition of claim 16, wherein the medical device is a vascular stent.

18. The composition of claim 1, wherein the polymer has a molecular weight in the range from about 15 000 Da to about 600 000 Da.

19. The composition of claim 1, wherein the composition further comprises at least one bioactive agent dispersed in the polymer.

20. The composition of claim 19, wherein the composition includes from about 5 to about 150 molecules of the bioactive agent per polymer molecule chain.

21. The composition of claim 19, wherein the bioactive agent is released from the composition under physiological conditions over a time selected from about 14 days to about 2 years.

22. The composition of claim 21, wherein the surgical device is an internal fixation device.

23. The composition of claim 21, wherein the surgical device is a dialysis shunt.

24. A method for delivering a bioactive agent to a subject in need thereof comprising implanting at an internal site in the subject a composition of claim 1 that further comprises a bioactive agent dispersed therein so that the composition biodegrades to deliver the bioactive agent to tissue surrounding the internal site at a controlled rate.

25. The method of claim 24, wherein the composition is contained within or at least partially coats a surgical device.

26. The method of claim 24, wherein the composition completely biodegrades within about seven days to about six months

27. A method for producing a biodegradable hydrogel comprising:
heating a mixture comprising a photoreactive polysaccharide and a derivative of a PEA of claim 1 that has been modified to contain stabilizing substituents at the oxirane cycle to produce a three-dimensional hydrogel.

28. The method of claim 27, wherein the mixture is cast onto a substrate to dry prior to the heating.

29. The method of claim 27, wherein the heat is from about 80° C. to about 120° C.

30. The method of claim 27, wherein the stabilizing substituents are active carbonate groups.

31. The method of claim 27, wherein the PEA is unsaturated.

32. The method of claim 27, wherein the PEA is compound PEA I.6.

33. The method of claim 27, wherein the method further comprises infusing the hydrogel with a water-based bioactive agent.

34. A biodegradable hydrogel produced by the method of any one of claim 27 to 33.
